(19) United States
(12) Patent Application Publication
(10) Pub. No.: US 2026/0167701 A1
(43) Pub. Date: Jun. 18, 2026

(54) MONOCLONAL ANTIBODY RECOGNIZING GP350 PROTEIN OF EPSTEIN-BARR VIRUS AND USE THEREOF

(71) Applicant: GUANGZHOU GENBIONOVA MEDICAL TECHNOLOGY CO., LTD., Guangzhou (CN)

(72) Inventors: Musheng ZENG, Guangzhou (CN); Gexin ZHAO, Guangzhou (CN); Penglin LI, Guangzhou (CN); Cong SUN, Guangzhou (CN); Boyu YUAN, Guangzhou (CN); Guokai FENG, Guangzhou (CN)

(73) Assignee: GUANGZHOU GENBIONOVA MEDICAL TECHNOLOGY CO., LTD., Guangzhou (CN)

(21) Appl. No.: 19/412,502

(22) Filed: Dec. 8, 2025

(30) Foreign Application Priority Data
Dec. 12, 2024 (CN) 202411836537.1

(51) Int. Cl. C07K 16/085; A61P 37/04

(52) U.S. Cl. CPC C07K 16/085; A61P 37/04; C07K 2317/76; C07K 2317/92

(57) ABSTRACT

The present disclosure belongs to the field of antibody technology, and discloses a monoclonal antibody recognizing gp350 protein of Epstein-Barr virus (EBV) and use thereof. The present disclosure provides a monoclonal antibody against EBV gp350 protein or an antigen-binding fragment thereof, comprising 1A12 and 2E4. 1A12 exhibits high affinity for the gp350 protein (KD(M)=3.415E⁻¹⁰), and 2E4 also exhibits high affinity for the gp350 protein (KD(M)=4.851E⁻⁹). These two monoclonal antibodies or antigen-binding fragments thereof can significantly inhibit EBV infection of epithelial cells and B cells, can be used for detecting the presence or level of gp350 protein in a sample, detecting EBV, diagnosing diseases caused by EBV infection, preventing EBV infection, and treating and preventing diseases caused by EBV infection.

### CROSS-REFERENCE TO RELATED APPLICATION

This application is based on and claims the benefit of priority from Chinese Patent Application No. 2024118365371, filed on Dec. 12, 2024, the entirety of which is incorporated by reference herein.

### INCORPORATION BY REFERENCE

This application includes a sequence listing in extensible Markup Language (a “xml” file) that is submitted herewith named P25GZ1NW00170US.xml created on Nov. 26, 2025, and 63,700 bytes in size. This sequence listing is incorporated by reference herein.

### TECHNICAL FIELD

The present disclosure belongs to the technical field of antibody technology, and specifically relates to a monoclonal antibody recognizing gp350 protein of Epstein-Barr virus and use thereof.

### BACKGROUND

Epstein-Barr virus (EBV), also known as human herpesvirus 4, is a known DNA oncogenic virus. It was first isolated by Epstein and Barr in 1964 from Burkitt lymphoma cells through in vitro suspension culture, and relevant cell lines were established. EBV belongs to the lymphotropic viruse of the gamma herpesvirus subfamily, the viral particle of which consists of four parts: core protein, capsid, tegument, and envelope, with the core protein carrying wrapped DNA.

EBV primarily exists in a latent infection state, with over 90% of infected individuals remaining in this state for life. The virus can be activated under specific conditions, which subsequently triggers carcinogenic effects. Current research indicates that EBV is closely associated with the development of various malignant tumors, including nasopharyngeal carcinoma, infectious mononucleosis, Hodgkin's lymphoma, non-Hodgkin's lymphoma, Burkitt's lymphoma, and various epithelial cell carcinomas including gastric cancer. The initial replication of EBV typically occurs in the oropharynx, growing and multiplying in B lymphocytes and oral epithelial cells, and then infecting B lymphocytes. These infected cells enter the bloodstream, causing systemic infection. When the immune function of host is compromised, latent EBV can be activated, leading to recurrent infection.

Despite the widespread health threats posed by EBV infection, there is currently no effective vaccine, and specific therapeutic methods for EBV-related diseases are also lacking. The treatment of infectious mononucleosis typically relies on antiviral drugs such as acyclovir. While these drugs can alleviate symptoms to a certain extent, they cannot completely eradicate EBV from B lymphocytes and throat epithelium. For EBV-related tumors, chemotherapy and radiotherapy remain the primary treatment modalities, however, the efficacies of chemotherapy and radiotherapy are often unsatisfactory in dealing with metastasis or recurrence.

Gp350 is one of the most major envelope glycoproteins of EBV and plays a key role in the viral infection process. As the most abundant protein on the EBV surface, gp350 primarily mediates the binding of virus to surface receptors on host cell (such as CD21, also known as CR2), initiating the viral infection process of B lymphocytes. Due to the critical function of gp350 in viral invasion, it has become an ideal target for vaccine development and antibody therapy. Gp350 antibodies effectively block the binding of EBV to B lymphocytes by specifically recognizing and binding to gp350, thereby preventing viral infection and viral spread. This blocking effect provides a theoretical basis for the use of gp350 antibodies in preventing primary infection and controlling reactivation of latent virus.

Although there are many studies on gp350, no relevant monoclonal antibodies have been widely applied in clinical practice yet. The main research focus is on developing highly effective gp350 antibodies to inhibit EBV infection and the occurrence of related diseases. Through genetic engineering techniques, gp350 antibodies with high affinity and specificity can be developed, thereby enhancing antiviral efficacy. These antibodies can not only be used for treating acute infections but also have the potential to serve as preventive treatments, especially in organ transplant recipients or immunocompromised individuals.

Monoclonal antibodies, due to their controllability in production, high affinity, high specificity, and significantly reduced adverse reactions in clinical applications, have become a highly promising approach in the treatment of infectious diseases. Through molecular engineering, the antiviral efficacy of these antibodies can be further improved. However, no monoclonal antibodies targeting EBV envelope glycoproteins have reached the market yet. Therefore, developing monoclonal antibodies against EBV will provide more effective means for preventing and treating EBV-related diseases.

### SUMMARY

The objective of a first aspect of the present disclosure is to provide a monoclonal antibody or an antigen-binding fragment thereof.

The objective of a second aspect of the present disclosure is to provide a recombinant protein.

The objective of a third aspect of the present disclosure is to provide a biological material related to the monoclonal antibody or the antigen-binding fragment thereof of the first aspect, or the recombinant protein of the second aspect of the present disclosure.

The objective of a fourth aspect of the present disclosure is to provide a conjugate.

The objective of a fifth aspect of the present disclosure is to provide use of the monoclonal antibody or the antigen-binding fragment thereof of the first aspect, the recombinant protein of the second aspect, the biological material of the third aspect, or the conjugate of the fourth aspect of the present disclosure in the preparation of a product.

The objective of a sixth aspect of the present disclosure is to provide a kit.

The objective of a seventh aspect of the present disclosure is to provide a drug.

The objective of an eighth aspect of the present disclosure is to provide a method for preparing the monoclonal antibody or the antigen-binding fragment thereof of the first aspect of the present disclosure.

To achieve the above objectives, the technical solutions used in the present disclosure are as follows.

The first aspect of the present disclosure provides a monoclonal antibody against EBV gp350 protein or an antigen-binding fragment thereof, wherein the monoclonal antibody or the antigen-binding fragment thereof comprises a heavy chain and a light chain.

In some embodiments of the present disclosure, the monoclonal antibody or the antigen-binding fragment thereof comprises any one of A) or B):
A) a monoclonal antibody or an antigen-binding fragment thereof designated as 1A12;
B) a monoclonal antibody or an antigen-binding fragment thereof designated as 2E4.
A) In the monoclonal antibody or the antigen-binding fragment thereof of 1A12, the heavy chain comprises:
a heavy chain variable region comprising CDR-H1, CDR-H2, and CDR-H3 of the heavy chain variable region, wherein the heavy chain variable region has an amino acid sequence shown in SEQ ID NO: 14;
the light chain comprises:
a light chain variable region comprising CDR-L1, CDR-L2, and CDR-L3 of the light chain variable region, wherein the light chain variable region has an amino acid sequence shown in SEQ ID NO: 28.

Preferably, amino acid sequences of CDR-H1, CDR-H2, CDR-H3, CDR-L1, CDR-L2, and CDR-L3 are as shown in SEQ ID NO: 18, SEQ ID NO: 19, SEQ ID NO: 20, SEQ ID NO: 31, SEQ ID NO: 32, and SEQ ID NO: 30, respectively, wherein the CDRs are defined according to the Kabat definition scheme.

Preferably, the amino acid sequences of CDR-H1, CDR-H2, CDR-H3, CDR-L1, and CDR-L3 are as shown in SEQ ID NO: 15, SEQ ID NO: 16, SEQ ID NO: 17, SEQ ID NO: 29, and SEQ ID NO: 30, respectively, and the amino acid sequence of CDR-L2 is: GAS, wherein the CDRs are defined according to the IMGT definition scheme.

Preferably, the amino acid sequences of CDR-H1, CDR-H2, CDR-H3, CDR-L1, CDR-L2, and CDR-L3 are as shown in SEQ ID NO: 21, SEQ ID NO: 22, SEQ ID NO: 20, SEQ ID NO: 31, SEQ ID NO: 32, and SEQ ID NO: 30, respectively, wherein the CDRs are defined according to the Chothia definition scheme.

Preferably, the amino acid sequences of CDR-H1, CDR-H2, CDR-H3, CDR-L1, CDR-L2, and CDR-L3 are as shown in SEQ ID NO: 23, SEQ ID NO: 24, SEQ ID NO: 25, SEQ ID NO: 33, SEQ ID NO: 34, and SEQ ID NO: 35, respectively, wherein the CDRs are defined according to the Contact definition scheme.

Preferably, the amino acid sequence of the heavy chain variable region comprises:
a1-A) SEQ ID NO: 14; or
a2-A) an amino acid sequence derived from SEQ ID NO: 14 by substitution, and/or deletion, and/or addition of one or several amino acids, and having the same function as a protein of SEQ ID NO: 14; or
a3-A) an amino acid sequence having 99%, 98%, 97%, 96%, 95%, 94%, 93%, 92%, 91%, 90%, 89%, 88%, 87%, 86%, 85%, 84%, 83%, 82%, 81%, or 80% homology to SEQ ID NO: 14, and having the same function as the protein of SEQ ID NO: 14.

The amino acid sequence of the light chain variable region comprises:
b1-A) SEQ ID NO: 28; or
b2-A) an amino acid sequence derived from SEQ ID NO: 28 by substitution, and/or deletion, and/or addition of one or several amino acids, and having the same function as a protein of SEQ ID NO: 28; or
b3-A) an amino acid sequence having 99%, 98%, 97%, 96%, 95%, 94%, 93%, 92%, 91%, 90%, 89%, 88%, 87%, 86%, 85%, 84%, 83%, 82%, 81%, or 80% homology to SEQ ID NO: 28, and having the same function as the protein of SEQ ID NO: 28.

Preferably, the heavy chain further comprises a heavy chain constant region; and the light chain further comprises a light chain constant region.

Preferably, an amino acid sequence of the heavy chain constant region comprises:
c1-A) an amino acid sequence consisting of amino acids 144-473 of SEQ ID NO: 13; or
c2-A) an amino acid sequence derived from the amino acid sequence of c1-A) by substitution, and/or deletion, and/or addition of one or several amino acids, and having the same function as a protein of the amino acid sequence of c1-A); or
c3-A) an amino acid sequence having 99%, 98%, 97%, 96%, 95%, 94%, or 93% homology to the amino acid sequence of c1-A), and having the same function as the protein of the amino acid sequence of c1-A).

Preferably, an amino acid sequence of the light chain constant region comprises:
d1-A) an amino acid sequence consisting of amino acids 128-233 of SEQ ID NO: 27; or
d2-A) an amino acid sequence derived from the amino acid sequence of d1-A) by substitution, and/or deletion, and/or addition of one or several amino acids, and having the same function as a protein of the amino acid sequence of d1-A); or
d3-A) an amino acid sequence having 99%, 98%, 97%, 96%, 95%, 94%, or 93% homology to the amino acid sequence of d1-A), and having the same function as the protein of the amino acid sequence of d1-A).

Preferably, the heavy chain further comprises a heavy chain signal peptide; and the light chain further comprises a light chain signal peptide.

Preferably, an amino acid sequence of the heavy chain signal peptide comprises:
e1-A) an amino acid sequence consisting of amino acids 1-19 of SEQ ID NO: 13; or
e2-A) an amino acid sequence derived from the amino acid sequence of e1-A) by substitution, and/or deletion, and/or addition of one or several amino acids, and having the same function as a protein of the amino acid sequence of e1-A); or
e3-A) an amino acid sequence having 99%, 98%, 97%, 96%, 95%, 94%, or 93% homology to the amino acid sequence of e1-A), and having the same function as the protein of the amino acid sequence of e1-A).

Preferably, an amino acid sequence of the light chain signal peptide comprises:
f1-A) an amino acid sequence consisting of amino acids 1-19 of SEQ ID NO: 27; or
f2-A) an amino acid sequence derived from the amino acid sequence of f1-A) by substitution, and/or deletion, and/or addition of one or several amino acids, and having the same function as a protein of the amino acid sequence of f1-A); or
f3-A) an amino acid sequence having 99%, 98%, 97%, 96%, 95%, 94%, or 93% homology to the amino acid sequence of f1-A), and having the same function as a protein of the amino acid sequence of f1-A).
B) In the monoclonal antibody or the antigen-binding fragment thereof of 2E4, the heavy chain comprises:
a heavy chain variable region comprising CDR-H4, CDR-H5, and CDR-H6 of the heavy chain variable region, wherein the heavy chain variable region has an amino acid sequence shown in SEQ ID NO: 38;
the light chain comprises:
a light chain variable region comprising CDR-L4, CDR-L5, and CDR-L6 of the light chain variable region, wherein the light chain variable region has an amino acid sequence shown in SEQ ID NO: 52.

Preferably, amino acid sequences of CDR-H4, CDR-H5, CDR-H6, CDR-L4, CDR-L5, and CDR-L6 are as shown in SEQ ID NO: 42, SEQ ID NO: 43, SEQ ID NO: 44, SEQ ID NO: 55, SEQ ID NO: 56, and SEQ ID NO: 54, respectively, wherein the CDRs are defined according to the Kabat definition scheme.

Preferably, the amino acid sequences of CDR-H4, CDR-H5, CDR-H6, CDR-L4, and CDR-L6 are as shown in SEQ ID NO: 39, SEQ ID NO: 40, SEQ ID NO: 41, SEQ ID NO: 53, and SEQ ID NO: 54, respectively, and the amino acid sequence of CDR-L5 is: RNN, wherein the CDRs are defined according to the IMGT definition scheme.

Preferably, the amino acid sequences of CDR-H4, CDR-H5, CDR-H6, CDR-L4, CDR-L5, and CDR-L6 are as shown in SEQ ID NO: 45, SEQ ID NO: 46, SEQ ID NO: 44, SEQ ID NO: 55, SEQ ID NO: 56, and SEQ ID NO: 54, respectively, wherein the CDRs are defined according to the Chothia definition scheme.

Preferably, the amino acid sequences of CDR-H4, CDR-H5, CDR-H6, CDR-L4, CDR-L5, and CDR-L6 are as shown in SEQ ID NO: 47, SEQ ID NO: 48, SEQ ID NO: 49, SEQ ID NO: 57, SEQ ID NO: 58, and SEQ ID NO: 59, respectively, wherein the CDRs are defined according to the Contact definition scheme.

Preferably, the amino acid sequence of the heavy chain variable region comprises:
a1-B) SEQ ID NO: 38; or
a2-B) an amino acid sequence derived from SEQ ID NO: 38 by substitution, and/or deletion, and/or addition of one or several amino acids, and having the same function as a protein of SEQ ID NO: 38; or
a3-B) an amino acid sequence having 99%, 98%, 97%, 96%, 95%, 94%, 93%, 92%, 91%, 90%, 89%, 88%, 87%, 86%, 85%, 84%, 83%, 82%, 81%, or 80% homology to SEQ ID NO: 38, and having the same function as the protein of SEQ ID NO: 38.

The amino acid sequence of the light chain variable region comprises:
b1-B) SEQ ID NO: 52; or
b2-B) an amino acid sequence derived from SEQ ID NO: 52 by substitution, and/or deletion, and/or addition of one or several amino acids, and having the same function as a protein of SEQ ID NO: 52; or
b3-B) an amino acid sequence having 99%, 98%, 97%, 96%, 95%, 94%, 93%, 92%, 91%, 90%, 89%, 88%, 87%, 86%, 85%, 84%, 83%, 82%, 81%, or 80% homology to SEQ ID NO: 52, and having the same function as the protein of SEQ ID NO: 52.

Preferably, the heavy chain further comprises a heavy chain constant region; and the light chain further comprises a light chain constant region.

Preferably, an amino acid sequence of the heavy chain constant region comprises:
c1-B) an amino acid sequence consisting of amino acids 139-468 of SEQ ID NO: 37; or
c2-B) an amino acid sequence derived from the amino acid sequence of c1-B) by substitution, and/or deletion, and/or addition of one or several amino acids, and having the same function as a protein of the amino acid sequence of c1-B); or
c3-B) an amino acid sequence having 99%, 98%, 97%, 96%, 95%, 94%, or 93% homology to the amino acid sequence of c1-B), and having the same function as the protein of the amino acid sequence of c1-B).

Preferably, an amino acid sequence of the light chain constant region comprises: d1-B) an amino acid sequence consisting of amino acids 130-235 of SEQ ID NO: 51; or
d2-B) an amino acid sequence derived from the amino acid sequence of d1-B) by substitution, and/or deletion, and/or addition of one or several amino acids, and having the same function as a protein of the amino acid sequence of d1-B); or
d3-B) an amino acid sequence having 99%, 98%, 97%, 96%, 95%, 94%, or 93% homology to the amino acid sequence of d1-B), and having the same function as the protein of the amino acid sequence of d1-B).

Preferably, the heavy chain further comprises a heavy chain signal peptide; and the light chain further comprises a light chain signal peptide.

Preferably, an amino acid sequence of the heavy chain signal peptide comprises:
e1-B) an amino acid sequence consisting of amino acids 1-19 of SEQ ID NO: 37; or
e2-B) an amino acid sequence derived from the amino acid sequence of e1-B) by substitution, and/or deletion, and/or addition of one or several amino acids, and having the same function as a protein of the amino acid sequence of e1-B); or
e3-B) an amino acid sequence having 99%, 98%, 97%, 96%, 95%, 94%, or 93% homology to the amino acid sequence of e1-B), and having the same function as the protein of the amino acid sequence of e1-B).

Preferably, an amino acid sequence of the light chain signal peptide comprises:
f1-B) an amino acid sequence consisting of amino acids 1-19 of SEQ ID NO: 51; or
f2-B) an amino acid sequence derived from the amino acid sequence of f1-B) by substitution, and/or deletion, and/or addition of one or several amino acids, and having the same function as a protein of the amino acid sequence of f1-B); or
f3-B) an amino acid sequence having 99%, 98%, 97%, 96%, 95%, 94%, or 93% homology to the amino acid sequence of f1-B), and having the same function as the protein of the amino acid sequence of f1-B).

In some embodiments of the present disclosure, the monoclonal antibody or the antigen-binding fragment thereof comprises at least one of a full-length antibody, Fab, Fab′, F(ab′)2, Fv, scFv, a bispecific antibody, or a multispecific antibody.

In some embodiments of the present disclosure, an amino acid sequence of EBV gp350 comprises:
g1) an amino acid sequence consisting of amino acids 11-817 of SEQ ID NO: 4; or
g2) an amino acid sequence derived from the amino acid sequence of g1) by substitution, and/or deletion, and/or addition of one or several amino acids, and having the same function as a protein of the amino acid sequence of g1); or
g3) an amino acid sequence having 99%, 98%, 97%, 96%, 95%, 94%, or 93% homology to the amino acid sequence of g1), and having the same function as the protein of the amino acid sequence of g1).

The second aspect of the present disclosure provides a recombinant protein comprising the monoclonal antibody or the antigen-binding fragment thereof of the first aspect of the present disclosure; and optionally a tag sequence facilitating expression and/or purification.

Preferably, the tag sequence is at least one selected from the group consisting of a His tag, a GGGS sequence (SEQ ID NO: 61), and a FLAG tag; further preferably a His tag; more preferably a 6×His tag.

The third aspect of the present disclosure provides a biological material related to the monoclonal antibody or the antigen-binding fragment thereof of the first aspect of the present disclosure, or the recombinant protein of the second aspect of the present disclosure, wherein the biological material comprises at least one of h1) to h16):
h1) a nucleic acid molecule encoding the monoclonal antibody or the antigen-binding fragment thereof of the first aspect, or the recombinant protein of the second aspect of the present disclosure;
h2) an expression cassette comprising the nucleic acid molecule of h1);
h3) a vector comprising the nucleic acid molecule of h1);
h4) a vector comprising the expression cassette of h2);
h5) a transgenic cell line comprising the nucleic acid molecule of h1);
h6) a transgenic cell line comprising the expression cassette of h2);
h7) a transgenic cell line comprising the vector of h3);
h8) a transgenic cell line comprising the vector of h4);
h9) a microorganism comprising the nucleic acid molecule of h1);
h10) a microorganism comprising the expression cassette of h2);
h11) a microorganism comprising the vector of h3);
h12) a microorganism comprising the vector of h4);
h13) a virus comprising the nucleic acid molecule of h1);
h14) a virus comprising the expression cassette of h2);
h15) a virus comprising the vector of h3);
h16) a virus comprising the vector of h4).

Preferably, the transgenic cell line does not comprise a propagating material.

Preferably, the nucleic acid molecule encoding the monoclonal antibody or the antigen-binding fragment thereof of the first aspect of the present disclosure comprises a nucleic acid molecule encoding the heavy chain of the monoclonal antibody or the antigen-binding fragment thereof of the first aspect of the present disclosure, and a nucleic acid molecule encoding the light chain of the monoclonal antibody or the antigen-binding fragment thereof of the first aspect of the present disclosure.

In some embodiments of the present disclosure, a nucleotide sequence of the nucleic acid molecule encoding the heavy chain of the monoclonal antibody or the antigen-binding fragment thereof of the first aspect of the present disclosure comprises:
A) for the monoclonal antibody or the antigen-binding fragment thereof of 1A12:
a211-A) a nucleotide sequence shown in SEQ ID NO: 26; or
a212-A) a nucleotide sequence derived from SEQ ID NO: 26 by substitution, and/or deletion, and/or addition of one or several nucleotides, and having the same function as a nucleic acid molecule of SEQ ID NO: 26; or
a213-A) a nucleotide sequence having no less than 80%, 85%, or 90% homology to SEQ ID NO: 26, and having the same function as the nucleic acid molecule of SEQ ID NO: 26.
B) for the monoclonal antibody or the antigen-binding fragment thereof of 2E4:
a211-B) a nucleotide sequence shown in SEQ ID NO: 50; or
a212-B) a nucleotide sequence derived from SEQ ID NO: 50 by substitution, and/or deletion, and/or addition of one or several nucleotides, and having the same function as a nucleic acid molecule of SEQ ID NO: 50; or
a213-B) a nucleotide sequence having no less than 80%, 85%, or 90% homology to SEQ ID NO: 50, and having the same function as the nucleic acid molecule of SEQ ID NO: 50.

In some embodiments of the present disclosure, a nucleotide sequence of the nucleic acid molecule encoding the light chain of the monoclonal antibody or the antigen-binding fragment thereof of the first aspect of the present disclosure comprises:
A) for the monoclonal antibody or the antigen-binding fragment thereof of 1A12:
a221-A) a nucleotide sequence shown in SEQ ID NO: 36; or
a222-A) a nucleotide sequence derived from SEQ ID NO: 36 by substitution, and/or deletion, and/or addition of one or several nucleotides, and having the same function as a nucleic acid molecule of SEQ ID NO: 36; or
a223-A) a nucleotide sequence having no less than 80%, 85%, or 90% homology to SEQ ID NO: 36, and having the same function as the nucleic acid molecule of SEQ ID NO: 36.
B) for the monoclonal antibody or the antigen-binding fragment thereof of 2E4:
a221-B) a nucleotide sequence shown in SEQ ID NO: 60; or
a222-B) a nucleotide sequence derived from SEQ ID NO: 60 by substitution, and/or deletion, and/or addition of one or several nucleotides, and having the same function as a nucleic acid molecule of SEQ ID NO: 60; or
a223-B) a nucleotide sequence having no less than 80%, 85%, or 90% homology to SEQ ID NO: 60, and having the same function as the nucleic acid molecule of SEQ ID NO: 60.

The fourth aspect of the present disclosure provides a conjugate comprising at least one of the monoclonal antibody or the antigen-binding fragment thereof of the first aspect of the present disclosure and the recombinant protein of the second aspect of the present disclosure; and
a conjugated moiety, wherein the conjugated moiety comprises at least one of a detectable label, a drug, a toxin, a cytokine, a radionuclide, or an enzyme.

Preferably, the detectable label is selected from the group consisting of a radioactive isotope, a fluorescent substance, a chemiluminescent substance, a colored substance, or any combination thereof.

Preferably, the conjugate is selected from the group consisting of a fluorescent substance, a chemiluminescent label, a colored substance, a radioactive isotope, an Magnetic Resonance Imaging (MRI) or Computed Tomography (CT) contrast agent, an enzyme capable of producing a detectable product, a radionuclide, a biological toxin, a cytokine (such as IL-2, etc.), an antibody, an antibody Fc fragment, an antibody scFv fragment, gold nanoparticles/nanorods, viral particles, liposomes, magnetic nanoparticles, a prodrug-activating enzyme, a chemotherapeutic agent (e.g., cisplatin), or any form of nanoparticles, etc.

The fifth aspect of the present disclosure provides use of the monoclonal antibody or the antigen-binding fragment thereof of the first aspect, the recombinant protein of the second aspect, the biological material of the third aspect, or the conjugate of the fourth aspect of the present disclosure in the preparation of a product;
wherein the product comprises at least one of a drug, a reagent, an assay plate, a kit, or a detection chip.

Preferably, the drug has at least one function of i1) to i2):
i1) preventing EBV infection; or
i2) treating and/or preventing diseases caused by EBV infection.

Preferably, the reagent, the assay plate, the detection chip, or the kit has at least one function of j1) to j3):
j1) detecting the presence or level of gp350 protein in a sample;
j2) detecting EBV; or
j3) diagnosing diseases caused by EBV infection.

Preferably, the diseases comprise at least one of nasopharyngeal carcinoma, gastric cancer, Hodgkin's lymphoma, Burkitt's lymphoma, NK/T cell lymphoma, lymphoproliferative disorders, or infectious mononucleosis.

Preferably, the drug comprises a vaccine.

The sixth aspect of the present disclosure provides a product comprising at least one of k1) to k3):
k1) the monoclonal antibody or the antigen-binding fragment thereof of the first aspect of the present disclosure;
k2) the recombinant protein of the second aspect of the present disclosure; or
k3) the conjugate of the fourth aspect of the present disclosure;
wherein the product comprises at least one of a reagent, an assay plate, a kit, or a detection chip.

Preferably, the product has at least one function of j1) to j3):
j1) detecting the presence or level of gp350 protein in a sample;
j2) detecting EBV; or
j3) diagnosing diseases caused by EBV infection.

Preferably, the diseases comprise at least one of nasopharyngeal carcinoma, gastric cancer, Hodgkin's lymphoma, Burkitt's lymphoma, NK/T cell lymphoma, lymphoproliferative disorders, or infectious mononucleosis.

The seventh aspect of the present disclosure provides a pharmaceutical composition comprising at least one of l1) to l4):
l1) the monoclonal antibody or the antigen-binding fragment thereof of the first aspect of the present disclosure;
l2) the recombinant protein of the second aspect of the present disclosure;
l3) the biological material of the third aspect of the present disclosure; or
l4) the conjugate of the fourth aspect of the present disclosure.

Preferably, the pharmaceutical composition further comprises a pharmaceutically acceptable carrier.

Preferably, the pharmaceutical composition has at least one function of i1) to i2):
i1) preventing EBV infection; or
i2) treating and/or preventing diseases caused by EBV infection.

Preferably, the diseases comprise at least one of nasopharyngeal carcinoma, gastric cancer, Hodgkin's lymphoma, Burkitt's lymphoma, NK/T cell lymphoma, lymphoproliferative disorders, or infectious mononucleosis.

Preferably, the pharmaceutical composition comprises a vaccine.

Preferably, a vaccine is provided, comprising at least one of l1) to l4) and an adjuvant:
l1) the monoclonal antibody or the antigen-binding fragment thereof of the first aspect of the present disclosure;
l2) the recombinant protein of the second aspect of the present disclosure;
l3) the biological material of the third aspect of the present disclosure; or
l4) the conjugate of the fourth aspect of the present disclosure.

The eighth aspect of the present disclosure provides a method for preparing the monoclonal antibody or the antigen-binding fragment thereof of the first aspect, or the recombinant protein of the second aspect of the present disclosure, comprising culturing the transgenic cell line, the microorganism, or the virus of the third aspect of the present disclosure.

The present disclosure has at least the following beneficial effects.

The present disclosure provides monoclonal antibodies against EBV gp350 or antigen-binding fragments thereof, including 1A12 and 2E4; wherein 1A12 has high affinity for the gp350 protein (KD(M)=3.415E⁻¹⁰), and 2E4 also has high affinity for the gp350 protein (KD(M)=4.851E⁻⁹). These two monoclonal antibodies or antigen-binding fragments thereof can significantly inhibit EBV infection of epithelial cells and B cells. They can be used for detecting the presence or level of gp350 protein in a sample, detecting EBV, diagnosing diseases caused by EBV infection, preventing EBV infection, and/or treating and/or preventing diseases caused by EBV infection.

### BRIEF DESCRIPTION OF DRAWINGS

FIG. 1 is a graph showing the affinity measurement result of monoclonal antibody 1A12 for the gp350 protein.

FIG. 2 is a graph showing the result of monoclonal antibody 1A12 blocking EBV infection of epithelial cells.

FIG. 3 is a graph showing the result of monoclonal antibody 1A12 blocking EBV infection of B cells.

FIG. 4 is a graph showing the affinity measurement result of monoclonal antibody 2E4 for the gp350 protein.

FIG. 5 is a graph showing the result of monoclonal antibody 2E4 blocking EBV infection of epithelial cells.

FIG. 6 is a graph showing the result of monoclonal antibody 2E4 blocking EBV infection of B cells.

### DETAILED DESCRIPTION

The concepts of and the resulting technical effects of the present disclosure will be clearly and completely described in conjunction with the examples in the following, so as to fully understand the objectives, features, and effects of the present disclosure. Apparently, the described examples are only part of the examples, rather than all of the examples of the present disclosure. Based on the examples of the present disclosure, all other examples obtained by those skilled in the art without creative efforts shall fall within the protection scope of the present disclosure.

### Example 1: Preparation of Monoclonal Antibodies (mAbs) Against Gp350 Protein of Epstein-Barr Virus (EBV Gp350)

1.1 Preparation of EBV gp350 Recombinant Protein

The gp350 protein plays an important role in the process of EBV invasion of epithelial cells and B cells. In the example of the present disclosure, the inventors selected gp350 as the bait protein to screen for specific antibodies.

The selected original sequence of gp350 included a KOZAK sequence, a signal peptide, a His tag, an extracellular segment of gp350 protein, a His tag, and a stop codon, which was specifically as follows: 5′-GCCACCATGGACGCCATGAAGAGGGGCCTGTGCTGCGTGCTGCTGCTGTGTGGCG CCGTGTTTGTGTCCCCCAGCCAGGAAATCCACGCCCGGTTCAGAAGAGGCGCCAG AggatccCACCACCATCATCACCATCATCACCACCATGAGGCAGCCTTGCTTGTGTGT CAGTACACCATCCAGAGCCTGATCCATCTCACGGGTGAAGATCCTGGTTTTTTCAA TGTTGAGATTCCGGAATTCCCATTTTACCCCACATGCAATGTTTGTACGGCAGATGT CAATGTAACTATCAATTTCGATGTCGGGGGCAAAAAGCATCAACTTGATCTTGACT TTGGCCAGCTGACACCCCATACGAAGGCTGTCTACCAACCTCGAGGTGCATTTGG TGGCTCAGAAAATGCCACCAATCTCTTTCTACTGGAGCTCCTTGGTGCAGGAGAAT TGGCTCTAACTATGCGGTCTAAGAAGCTTCCAATTAACGTCACCACCGGAGAGGA GCAACAAGTAAGCCTGGAATCTGTAGATGTCTACTTTCAAGATGTGTTTGGAACCA TGTGGTGCCACCATGCAGAAATGCAAAACCCCGTGTACCTGATACCAGAAACAGT GCCATACATAAAGTGGGATAACTGTAATTCTACCAATATAACGGCAGTAGTGAGGG CACAGGGGCTGGATGTCACGTTACCCTTAAGTTTGCCAACGTCAGCTCAAGACTC GAATTTCAGCGTAAAAACACAAATGCTCGGTAATGAGATAGATATTGAGTGTATTAT GGAGGATGGCGAAATTTCACAAGTTCTGCCCGGAGACAACAAATTTAACATCACC TGCAGTGGATACGAGAGCCATGTTCCCAGCGGCGGAATTCTCACATCAACGAGTC CCGTGGCCACCCCAATACCTGGTACAGGGTATGCATACAGCCTGCGTCTGACACCA CGTCCAGTGTCACGATTTCTTGGCAATAACAGTATCCTGTACGTGTTTTACTCTGGG AATGGACCGAAGGCGAGCGGGGGAGATTACTGCATTCAGTCCAACATTGTGTTCT CTGATGAGATTCCAGCTTCACAGGACATGCCGACAAACACCACAGACATCACATA TGTGGGTGACAATGCTACCTATTCAGTGCCAATGGTCACTTCTGAGGACGCAAACT CGCCAAATGTTACAGTGACTGCCTTTTGGGCCTGGCCAAACAACACTGAAACTGA CTTTAAGTGCAAATGGACTCTCACCTCGGGGACACCTTCGGGTTGTGAAAATATTT CTGGTGCATTTGCGAGCAATCGGACATTTGACATTACTGTCTCGGGTCTTGGCACG GCCCCCAAAACACTCATTATCACACGAACGGCTACCAATGCCACCACAACAACCC ACAAGGTTATATTCTCCAAGGCACCCGAGAGCACCACCACCTCCCCTACCTTGAAT ACAACTGGATTTGCTGATCCCAATACAACGACAGGTCTACCCAGCTCTACTCACGT GCCTACCAACCTCACCGCACCTGCAAGCACAGGCCCCACTGTATCCACAGCGGAT GTCACCAGCCCAACACCAGCCGGCACAACGTCAGGCGCATCACCGGTGACACCA AGTCCATCTCCACGGGACAACGGCACAGAAAGTAAGGCCCCCGACATGACCAGC CCCACATCAGCAGTGACTACCCCAACCCCAAATGGCACCAGCCCCACCCCAGCAA TGACTACCCCAACCCCAAATGCCACCAGCCCCACCTTGGGAAAAACAAGTCCCAC CTCAGCAGTGACTACCCCAACCCCAAATGCCACCAGCCCCACCCCAGCAGTGACT ACCCCAACCCCAAATGCCACCAGCCCTACTGTGGGAGAAACAAGTCCACAGGCA AATGCCACCAACCACACCTTAGGAGGAACAAGTCCCACCCCAGTAGTTACCAGCC CACCAAAAAATGCAACCAGTGATGTTACCACAGGCCAACATAACAGAACTTCAAG TTCAACCTCTTCCATGTCACTGAGACCCAGTTCAATCCCAGAGACAACGTCACATA TGCCTTTACTAACCTCCGCTCACCCAACAGGTGGTGAAAATATAACACAGGTGAC ACCAGCCTCTATCAGCACACATCATGTGTCCACCAGTTCGCCAGCGCCCCGCCCA GGCACCACCAGCCAAGCGTCAGGCCCTGGAAACAGTTCCACATCCACAAAACCG GGGGAGGTTAATGTCACCAAAGGCACGCCCCCCAAAAATGCAACGTCGCCCCAG GCCCCCAGTGGCCAAAAGACGGCGGTTCCCACGGTCACCTCAACAGGTGGAAAG GCCAATTCTACCACCGGTGGAAAGCACACCACAGGACATGGAGCCCGGACAAGT ACAGAGCCCACCACAGATTACGGCGATGATTCAACTACGCCAAGACCGAGATACA ATGCGACCACCTATCTACCTCCCAGCACTTCTAGCAAACTGCGGCCCCGCTGGACT TTTACGAGCCCACCGGTTACCACAGCCCAAGCCACCGTGCCTGTCCCGCCAACGT CCCAGCCCAGATTCTCAAACCTCCACCACCATCATCACCATCATCACCACCATTAA-3′ (SEQ ID NO: 1); wherein, the sequence consisting of nucleotides 1-6 of SEQ ID NO: 1 was the KOZAK sequence, the sequence consisting of nucleotides 7-117 of SEQ ID NO: 1 was the signal peptide sequence; the sequence consisting of nucleotides 118-147 of SEQ ID NO: 1 was the His tag sequence, the sequence consisting of nucleotides 148-2568 of SEQ ID NO: 1 was the extracellular segment sequence of gp350 protein, the sequence consisting of nucleotides 2569-2598 of SEQ ID NO: 1 was the His tag sequence, and the sequence consisting of nucleotides 2599-2601 of SEQ ID NO: 1 was the stop codon.

The above original sequence of gp350 was ligated into the mammalian cell expression vector pcDNA3.1+(Invitrogen), the specific method was as follows:

### (1) Amplification of the Gene of Gp350 Protein:

The above original sequence of gp350 was amplified using a 50 μL NEB Q5® PCR amplification reaction system (Table 1), wherein the upstream primer was: 5′-GAGGCAGCCTTGCTTGTGTG-3′ (SEQ ID NO: 2); the downstream primer was: 5′-GAGGTTTGAGAATCTGGGCTGG-3′ (SEQ ID NO: 3).

The amplified target fragment was analyzed by agarose gel electrophoresis, and the band with a correct molecular weight was cut under UV light. The PCR product was recovered according to the instruction of the commercial kit.

TABLE 1
PCR Amplification Reaction System
for the Original Sequence of gp350

| Component | Amount | |
|---|---|---|
| 5 × Reaction Buffer | 10 | μL |
| dNTPs | 1 | μL |
| Upstream Primer (10 μM) | 2.5 | μL |
| Downstream Primer (10 μM) | 2.5 | μL |
| EBV Genomic DNA Template | 1 | μL |
| DNA Polymerase | 0.5 | μL |
| High GC Enhancer (from NEB Q5 Kit) | 10 | μL |

| | |
|---|---|
| ddH₂O | Make up to 50 μL |

### (2) Restriction Digestion and Ligation of the Target Fragment and Vector:

The eukaryotic expression plasmid pcDNA3.1+ was used a vector. Both the target fragment and the vector were digested with NotI and BglII (a 50 μL reaction system for restriction digestion (Table 2) was used for digestion).

TABLE 2
Reaction System for Restriction Digestion

| Component | Amount |
|---|---|
| 10 × CutSmart Buffer | 5 μL |
| BglII-HF | 1 μL |
| NotI-HF | 1 μL |
| Target Fragment or Vector | 5 μg |
| ddH₂O | Make up to 50 μL |

Restriction digestion was performed at 37° C. for 2-3 h. The digested vector was run on an agarose gel and recovered using a gel recovery kit to obtain a linearized vector. The inserted fragment was directly recovered using a DNA purification kit. After recovery, ligation was performed using a 10 μL ligation reaction system (Table 3). The reaction condition was 37° C. for 30 min. The ligated product was obtained.

TABLE 3
Ligation Reaction System

| Component | Amount | |
|---|---|---|
| 5 × CEII Buffer | 2 | μL |
| CEII | 1 | μL |
| Linearized Vector | 100 | ng |
| Inserted Fragment | 100 | ng |

| | |
|---|---|
| ddH₂O | Make up to 10 μL |

### (3) Transformation of the Ligated Product and Screening of Positive Clones:

The ligated product was added to freshly thawed DH5α competent cell suspension, placed on ice for 30 min, heat-shocked at 42° C. for 90 s, and placed back on ice for 5 min. Then, 200 μL of LB medium was added to a resulting suspension, and the cells were resuscitated by slow shaking at 30° C. for 40 min. A resulting culture was spread on an ampicillin resistant LB plate and incubated at 37° C. overnight.

Single clone colonies were picked for sequencing verification. After the sequencing results were confirmed to be correct, the target recombinant plasmid was obtained. The plasmid was extracted in large quantities.

### (4) Expression and Extraction of Recombinant Protein:

Human kidney epithelial 293F cells were cultured to obtain 1 L of cell suspension with a density of 1.5×10⁶ cells/mL. The recombinant plasmid obtained in step (3) was transfected using a PEI transfection reagent. The specific procedures were as follows: 2 mg of the recombinant plasmid was diluted with 25 mL of Union-293 medium; and 6 mL of 1 mg/mL PEI was added to another 25 mL of Union-293 medium. The plasmid and PEI were mixed thoroughly by vortex, left at room temperature for 20 min, and then added to the 293F cell suspension. After 5 days of culture, the cell supernatant was harvested, centrifuged at 6000 rpm for 1 h at 4° C., and the cell pellet was discarded to obtain the supernatant containing the target protein.

The target protein was purified using affinity chromatography.

Since the resulting gp350 recombinant protein carried a 10×His tag, affinity purification could be performed using a nickel column. The specific procedures were as follows: the supernatant containing the target protein as obtained above was filtered through a 0.65 m filter paper, and passed through nickel column with beads for three times for binding, the beads were washed three times with 30 mM imidazole, and the target protein was eluted with 500 mM imidazole. Gel filtration chromatography was then used for further purification, the specific procedures were as follows: the protein eluted from the nickel column was concentrated using a 3 kD concentrator until the volume was less than 1 mL. Then, a resulting concentrated protein was purified using a Superdex 200 Increase 10/300 GL column.

The finally obtained amino acid sequence of the target protein was: HHHHHHHHHHEAALLVCQYTIQSLIHLTGEDPGFFNVEIPEFPFYPTCNVCTADVNVT INFDVGGKKHQLDLDFGQLTPHTKAVYQPRGAFGGSENATNLFLLELLGAGELAL™ RSKKLPINVTTGEEQQVSLESVDVYFQDVFGTMWCHHAEMQNPVYLIPETVPYIKW DNCNSTNITAVVRAQGLDVTLPLSLPTSAQDSNFSVKTQMLGNEIDIECIMEDGEISQV LPGDNKFNITCSGYESHVPSGGILTSTSPVATPIPGTGYAYSLRLTPRPVSRFLGNNSILY VFYSGNGPKASGGDYCIQSNIVFSDEIPASQDMPTNTTDITYVGDNATYSVPMVTSED ANSPNVTVTAFWAWPNNTETDFKCKWTLTSGTPSGCENISGAFASNRTFDITVSGLGT APKTLIITRTATNATTTTHKVIFSKAPESTTTSPTLNTTGFADPNTTTGLPSSTHVPTNLT APASTGPTVSTADVTSPTPAGTTSGASPVTPSPSPRDNGTESKAPDMTSPTSAVTTPTP NGTSPTPAMTTPTPNATSPTLGKTSPTSAVTTPTPNATSPTPAVTTPTPNATSPTVGETSP QANATNHTLGGTSPTPVVTSPPKNATSDVTTGQHNRTSSSTSSMSLRPSSIPETTSHMP LLTSAHPTGGENITQVTPASISTHHVSTSSPAPRPGTTSQASGPGNSSTSTKPGEVNVTK GTPPKNATSPQAPSGQKTAVPTVTSTGGKANSTTGGKHTTGHGARTSTEPTTDYGDD STTPRPRYNATTYLPPSTSSKLRPRWTFTSPPVTTAQATVPVPPTSQPRFSNLHHHHHH HHHH (SEQ ID NO: 4); wherein, the sequence consisting of amino acid residues 11-817 of SEQ ID NO: 4 was the extracellular segment sequence of gp350 protein, and the sequences consisting of amino acid residues 1-10 and 818-827 were the His tag sequences.

### 1.2. Construction of Phage Antibody Library

### (1) Total RNA Extraction:

Blood of nasopharyngeal carcinoma (NPC) patients was diluted 1:1 with PBS (10 mL blood+10 mL PBS). Then, 20 mL of the diluted blood was carefully layered over 15 mL of lymphocyte separation medium, and the layered interface was maintained. Centrifugation was performed at 2000 rpm, room temperature for 20 min, with slow deceleration. The middle mononuclear cell layer was aspirated into a new 15 mL centrifuge tube, then added with PBS to a final volume of 15 mL, and followed by centrifugation at 300 g, room temperature for 20 min. The supernatant was transferred to a new 50 mL centrifuge tube and centrifuged at 300 g, room temperature for 20 min. The supernatant was discarded. The cell pellets from both the 15 mL and 50 mL centrifuge tubes were each resuspended with 1 mL of Trizol (i.e., each 10 mL of blood was resuspended with 2 mL Trizol).

4 mL of chloroform was added to 20 mL of the Trizol-cell pellet resuspension. A resulting mixture was vortexed in a vibrator for 15 s and then left to stand at room temperature for 5 min. Centrifugation was performed at 4000 g, 4° C. for 30 min, layers were formed after centrifugation. The top transparent layer was carefully transferred with a pipette to a new RNase- and DNase-free 50 mL centrifuge tube. An equal volume of isopropanol was added to each tube, mixed thoroughly by inverting for several times, and left to stand at room temperature for 10 min. Centrifugation was performed at 4000 g for 30 min to remove the supernatant and obtain the pellet. 1 mL of 75% ethanol was added to the pellet, a resulting mixture was then transferred to a 1.5 mL centrifuge tube. The pellet was flicked multiple times to ensure full contact with the liquid. Centrifugation was performed at 7500 g for 5 min to remove the supernatant and obtain the pellet. With the tube cap open, the pellet was air-dried at room temperature for 10 min. 400 μL of nuclease-free water was then added to the pellet, and the tube was incubated at 55° C. for 10 min to ensure complete dissolution of RNA to obtain total RNA.

1 μL of the total RNA was taken to measure RNA concentration and A260/A280 ratio with a nucleic acid concentration measuring instrument and recorded.

### (2) RNA Reverse Transcription:

The total RNA obtained in step (1) was reverse transcribed using a reverse transcription kit (Promega GoScript™ Reverse Transcription Kit).

Specific procedures were as follows:

The above total RNA sample was divided into two parts, with one part using the Oligo dT Primer provided in the kit as the primer, and the other part using the Random 6-mers provided in the kit as the primer. The reaction mixture was prepared in a 1.5 mL centrifuge tube according to the system shown in Table 4, and the reaction system was proportionally scaled up based on the amount of RNA.

The mixture after reaction was aliquoted into 8-strip PCR tubes and placed in a PCR machine, briefly centrifuged, reacted at 70° C. for 5 min to denature the RNA, and immediately cooled on ice after reaction. Then, other reaction components were added to the above 8-strip PCR tubes according to the proportions in Table 5. A resulting reaction mixture from Table 5 was thoroughly mixed and briefly centrifuged, and the tubes were placed in the PCR machine. A reaction was performed at 45° C. for 60 min, then at 75° C. for 15 min, followed by cooling on ice, to obtain cDNA after reverse transcription of the total RNA, which was stored at 4° C.

TABLE 4
5 μL Reaction System for Reverse Transcription

| Component | Amount |
|---|---|
| Oligo dT Primer (50 μM)/Random 6-mers (50 μM) | 1 μL |
| Total RNA | 2 μg |
| ddH₂O | Make up to 5 μL |

TABLE 5
Proportions of Other Reaction Components

| Component | Amount | |
|---|---|---|
| Post-reaction system from Table 4 | 5 | μL |
| 5 × Reaction Buffer | 4 | μL |
| MgCl₂ | 2 | μL |
| dNTP mixture | 1 | μL |
| RNase Inhibitor | 0.4 | μL |
| RTase | 1 | μL |

| | |
|---|---|
| ddH₂O | Make up to 20 μL |

### (3) PCR Amplification:

The cDNA obtained in step (2) was used as a template to amplify single-chain antibody fragments (ScFv) by two rounds of PCR using NEB Q5 High-Fidelity DNA Polymerase. The constructed structure of the ScFv was VL-linker-VH (light chain-linker-heavy chain).

The first-round PCR was performed by using the cDNA as a template. Reaction system for the first-round PCR was shown in Table 6. The PCR reaction employed a three-temperature program, with each cycle consisting of denaturation at 95° C. for 10 s, annealing at 60° C. for 30 s, and extension at 72° C. for 1 min, for a total of 35 cycles. The primers for the first-round PCR comprised:

Vλ Forward Primer:
(SEQ ID NO: 5)
5′-CCTTTCTATGCGGCCCAGCCGGCCgagctcCAGTCT
GTSBTGACGCAGCCGCC-3′;
Vλ-linker Reverse Primer:
(SEQ ID NO: 6)
5′-GGAAGATCTAGAGGAACCACCTAGGACGGTSASCTT
GGTCC-3′;
Vκ Forward Primer:
(SEQ ID NO: 7)
5′-CCTTTCTATGCGGCCCAGCCGGCCgagctcGACATC
CRGDTGACCCAGTCTCC-3′;
Vκ-linker Reverse Primer:
(SEQ ID NO: 8)
5′-GGAAGATCTAGAGGAACCACCTTTGATTTCCACCTT
GGTCC-3′;
linker-VH Forward Primer:
(SEQ ID NO: 9)
5′-GGTGGTTCCTCTAGATCTTCCTCCTCTGGTGGCGGT
GGCTCGGGCGGTGGTGGGCAGGTGCAGCTGCAGGAGTCS
G-3′;
linker-VH Reverse Primer:
(SEQ ID NO: 10)
5′-CAGTCATTCTCGACTTactagtTGAGGAGACRGTGA
CCAGGGTG-3′.

Wherein, the lowercase section in each primer was restriction sites; the VX Forward Primer and the VX-linker Reverse Primer were used as a pair; the Vx Forward Primer and the Vx-linker Reverse Primer were used as a pair, and the linker-VH Forward Primer and the linker-VH Reverse Primer were used as a pair.

TABLE 6
Reaction System for the First-Round PCR

| Component | Amount | |
|---|---|---|
| cDNA | 5 | μL |
| Forward Primer (10 μM) | 2.5 | μL |
| Reverse Primer (10 μM) | 2.5 | μL |
| dNTP Mixture | 1 | μL |
| 5 × Reaction Buffer | 10 | μL |
| High GC Enhancer | 10 | μL |
| Polymerase | 0.5 | μL |

| | |
|---|---|
| ddH₂O | Make up to 50 μL |

After the reaction, all PCR products were subjected to 1.5% agarose gel electrophoresis. Bands of the target fragment sizes of around 320 bp (corresponding to VL) and 350 bp (corresponding to VH) were cut and recovered. DNA recovery was performed using the NEB DNA GEL purification kit according to the instruction of the kit. The collected DNA solution was the amplification product of the first-round PCR, which was measured for concentration and stored at 4° C.

The amplification product of the first-round PCR was used as a template of the second-round PCR. Reaction system for the second-round PCR was shown in Table 7. Reaction conditions for the second-round PCR were the same as those for the first-round PCR. Primers for the second-round PCR comprised: OF: 5′-CCTTTCTATGCGGCCCAGCCGGCCgagctc-3′ (SEQ ID NO: 11); OR: 5′-CAGTCATTCTCGACTTactagt-3′ (SEQ ID NO: 12).

TABLE 7
Reaction System for the Second-Round PCR

| Component | Amount | |
|---|---|---|
| VH/VL Gel-Recovered Product | 80 ng VH + 80 ng VL | |
| dNTP Mixture | 1 | μL |
| 5 × Reaction Buffer | 10 | μL |
| High GC Enhancer | 10 | μL |
| Forward Primer OF (10 μM) | 2.5 | μL |
| Reverse Primer OR (10 μM) | 2.5 | μL |
| Polymerase | 0.5 | μL |

| | |
|---|---|
| ddH₂O | Make up to 50 μL |

After the reaction, the PCR products were subjected to 1.5% agarose gel electrophoresis at 160V for 20 min. Finally, bands of the target fragment size of 750 bp were selected from the electrophoresis. DNA recovery was performed using the NEB DNA GEL purification kit according to the instruction of the kit. The collected DNA solution was the amplification product of the second-round PCR, which was measured for concentration and stored at 4° C.

### (4) Restriction Digestion and Ligation of Vector and the PCR Amplification Product:

The second-round PCR product (ScFv fragment) were ligated into phage plasmid pComb3XSS via restriction digestion, thereby constructing a phage plasmid library containing the amplified target fragment (ScFv fragment). Restriction enzymes SpeI and SacI were used for the restriction digestion.

The restriction digestion systems for the pComb3XSS vector and the second-round PCR amplification product (amplified target fragment (ScFv fragment)) were shown in Table 8 and Table 9, respectively.

TABLE 8
Restriction Digestion System for the Vector

| Component | Amount |
|---|---|
| Vector (pComb3XSS) | 20 μg |
| Spel | 10 μL |
| SacI | 10 μL |
| 10 × CutSmart Buffer | 50 μL |
| Quick CIP | 5 μL |
| ddH₂O | Make up to 500 μL |

TABLE 9
Restriction Digestion System for the
Target Fragment (ScFv Fragment)

| Component | Amount | |
|---|---|---|
| scFv | 5 | μg |
| SpeI | 5 | μL |
| SacI | 5 | μL |
| CutSmart 10 × buffer | 25 | μL |

| | |
|---|---|
| ddH₂O | Make up to 250 μL |

Reaction conditions for the restriction digestion: the systems were incubated at 37° C. for 2 h, and then at 80° C. for 3 min. After the restriction digestion, the vector digestion product was run on a gel, and the gel was cut and recovered using the NEB DNA purification and recovery kit (UV exposure was avoided). The digestion product of ScFv fragment was not run on a gel and could be recovered directly.

After purification of the digestion products, concentrations of the recovered products were measured. Then, the digested vector and the ScFv digestion product were ligated according to the ligation reaction system shown in Table 10. The above ligation system was reacted at 37° C. overnight (16-24 h). After ligation, the ligation products were recovered using the NEB DNA purification and recovery kit. Concentrations of the recovered ligation products were measured and recorded, and the recovered ligation products were stored at 4° C.

TABLE 10
Ligation Reaction System

| Component | Amount | |
|---|---|---|
| 10 × T4 reaction Buffer | 2 | μL |
| ScFv Fragment (digested) | 68 | ng |
| Vector (digested) | 100 | ng |
| T4 Ligase | 1 | μL |

| | |
|---|---|
| ddH₂O | Make up to 20 μL |

### 1.3. Construction of Bacterial Library:

(1) Preparation of TG1 E. coli Competent Cells:

TG1 strain was streaked on a 2×YT solid medium for single colonies and cultured overnight at 37° C. A single colony was picked from the single colony plate into 10 mL of 2×YT medium and cultured overnight at 37° C., 220 rpm. The bacteria were inoculated into 100 mL of 2×YT medium at a 1:100 dilution ratio, cultured at 37° C., 250 rpm for 40 min, and then measured the OD value. Thereafter, measurements were performed every 20 min until OD₆₀₀=0.3-0.35. The bacterial culture was collected, centrifuged at 3200 g, 0-4° C. for 10 min, and the supernatant was discarded. The pellet was placed on ice, resuspended in 40 mL of pre-chilled ddH₂O, centrifuged at 3200 g, 0-4° C. for 10 min, and the supernatant was discarded. Then, the pellet was placed on ice, resuspended in 1 mL of pre-chilled ddH₂O, transferred to a pre-chilled 1.5 mL EP tube, centrifuged at 10000 g, 4° C. for 30 s, and the step was repeated once. The supernatant was discarded, the pellet was placed on ice, and resuspended in 400 μL of pre-chilled ddH₂O, so as to obtain a suspension of TG1 E. coli competent cells.

The pComb3XSS recombinant plasmid containing the ScFv fragment obtained in the above example was used to construct an E. coli library by electroporation transformation method. The specific procedures were as follows: 100 ng of the pComb3XSS recombinant plasmid containing the ScFv fragment was added to 50 μL of the above suspension of TG1 E. coli competent cells using pre-chilled pipette tips, mixed gently by pipetting, and transferred to a pre-chilled 1 mm electroporation cuvette. After confirming that the mixture was at the bottom of the cuvette and was free of bubbles, electroporation was performed at 1800V, 1 mm gap. After the electroporation was completed, 1000 μL of 37° C. SOC medium was immediately added, and a resulting mixture was removed from the electroporation cuvette and resuscitated by shaking at 37° C., 180 rpm for 90 min. 10-fold gradient dilutions were performed using 2×YT liquid medium with a total of 6 dilution gradients (diluted for 101, 10², 10³, 104, 105, 10⁶ times, respectively). 5 μL from each gradient was evenly dropped onto 2×YT-GA solid medium, air-dried, and then statically cultured at 37° C. overnight. The number of colonies on the gradient dilution plates was counted, and the ligation efficiency was calculated. The formula for calculating ligation efficiency was:

$$E ( pfu / μg ) = N \times D \times 10 ;$$

wherein, E represented competent cell efficiency (unit: pfu/μg), D represented dilution factor, and N represented the number of single colonies on the plate corresponding to that dilution factor.

Electroporation transformation reaction was repeated for 100 times according to the above method. Resuscitated bacterial cultures were evenly spread onto 100 2×YT-GA 245 mm square plates with culture medium. After air-drying, the plates were incubated inverted overnight at 37° C. The overnight-incubated square plates were taken, 6 mL of 2×YT liquid medium was added to the surface of each plate, colonies from all the 100 square plates were gently scraped off using a spreader rod and bacterial culture was collected into a 50 mL centrifuge tube. Glycerol was added to a final concentration of 20% to obtain a bacterial library.

10 μL of the bacterial library was added to 990 μL of 2×YT liquid medium, and the OD₆₀₀ was measured using NanoDrop. The total OD₆₀₀ of the bacterial library was calculated and recorded.

$${T}_{OD 600} = {M}_{OD 600} \times 100 ;$$

wherein T_{OD600} represented total bacterial OD₆₀₀, and M_{OD600} represented measured OD₆₀₀.

### 1.4. Construction of Phage Library:

An appropriate amount of the above bacterial library was taken into a 1.5 mL EP tube. The volume of bacterial culture was calculated according to the formula as follows:

$$V ( μ L ) = 10 / {OD}_{600} \times 1 \; 000 ;$$

wherein V represented a volume of transferred bacterial culture (in μL), and OD₆₀₀ represented the total OD₆₀₀ of the constructed bacterial library.

The bacterial library was transferred into 100 mL of 2×YT-GA liquid medium to achieve an initial OD₆₀₀ of 0.1. The bacteria were cultured in a shaker with constant temperature at 37° C., 250 rpm until OD₆₀₀ of the bacterial culture reached 0.5-0.55. Helper phage M13K07 was calculated and added according to the following formula to achieve a bacteria: phage ratio of 1:20. Volume of the added helper phage was calculated according to the following formula:

$$V ( mL ) = ( 8 \times 1 {0}^{11} ) / {T}_{helper - phage} ;$$

wherein V represented the volume of the added helper phage (in mL), T_{helper-phage} represented titer of the helper phage used, and OD₆₀₀ represented OD₆₀₀ value of the bacterial culture.

Cultivation was continued in a shaker with constant temperature at 37° C., 220 rpm for 30 min. TG1 bacteria were collected by centrifugation at 3200 g for 5 min, the supernatant was discarded, and the pellet was resuspended and transferred to 100 mL of 2×YT-AK liquid medium, which was then cultured overnight in a shaker with constant temperature at 30° C., 250 rpm.

The overnight-cultured bacterial culture was transferred to a new 50 mL centrifuge tube and centrifuged at 4000 g, 4° C. for 30 min. The supernatant was harvested, ¼ volume of 20% PEG/2.5M NaCl pre-chilled at 4° C. was added, mixed thoroughly, and placed on ice for 30 min. Centrifugation was performed at 4000 rpm, 4° C. for 30 min, the supernatant was discarded, and the tube was inverted on paper for 2 min. The pellet was resuspended in 1 mL PBS, centrifuged at 12000 rpm, 4° C. for 20 min. The supernatant was harvested, ¼ volume of pre-chilled 20% PEG/2.5M NaCl solution was added, mixed evenly, and placed on ice for 10 min. Centrifugation was performed at 12000 rpm, 4° C. for 10 min, the supernatant was discarded, and the pellet was resuspended in 1 mL PBS. Centrifugation was performed at 12000 rpm, 4° C. for 2 min, and the supernatant was harvested, so as to obtain a phage library. Glycerol was added to a final concentration of 20% and stored at −80° C.

Titer of the phage library was determined. The specific procedures were as follows: the phage library was added to 10 mL of 2×YT liquid medium and cultured at 37° C., 250 rpm for about 45-60 min until OD₆₀₀ reached 0.5-0.55. 10 μL of the cultured phage library was taken and 10-fold gradient dilutions were performed (diluted for 13 gradients in total). 90 μL of untreated TG1 bacterial culture was added to each dilution gradient, mixed evenly, and incubated at 37° C. for 20 min. Then, 5 μL from each dilution gradient was dropped onto 2×YT-GA solid medium, air-dried, and cultured overnight at 37° C. Colonies were counted, and the number of phage particles per mL of phage solution was calculated according to the phage library titer formula as follows:

$$T ( pfu / mL ) = N \times D \times 400 ;$$

wherein T represented phage titer (in pfu/mL), D represented dilution factor, and N represented the number of single colonies corresponding to that dilution factor.

### 1.5. Screening for Gp350 Protein-Specific Antibodies

Solid-phase adsorption of antigen for screening was used to screen for antibodies that specifically bind to the gp350 protein.

The specific procedures were as follows:

50 g of the gp350 protein obtained above was dissolved in 2 mL PBS, and was used for coating an immune-tube overnight at 4° C. (positive tube). Meanwhile, a negative control protein (50 μg BSA) was used for coating as a negative control. The protein for coating was discarded, the tube was rinsed 3 times with 2 mL PBS, and then blocked with 2 mL of 3% BSA (dissolved in PBST) at room temperature for 2 h. The blocking solution was discarded. 100 μL of the obtained phage library was diluted with 2 mL PBS and incubated in the immune-tube for 1 h. The liquid was discarded, and the tube was washed with 2 mL PBST for 5 times, 5 min each time. Then, the tube was washed with 2 mL PBS for 5 times, 5 min each time. During washing, 1 mL of saturated TG1 bacterial culture overnight was added to 100 mL of 2×YT liquid medium and cultured at 37° C., 150 rpm until OD₆₀₀=0.5 (approximately 1.5 h). The washing solution was discarded, and elution was performed with 1 mL of 0.1 mg/mL Trypsin at room temperature for 30 min. 1 mL of eluate was added to 10 mL of the prepared TG1 bacterial culture (OD=0.5) and infected at 37° C., 150 rpm for 30 min, and centrifugation was performed at 4° C., 3000 g for 10 min. The pellet was then resuspended with 1 mL of 2×YT liquid medium. 1 mL of resuspended product eluted from the positive immune-tube was evenly spread on a 245 mm×245 mm 2×YT solid culture dish and cultured overnight at 37° C. for 16-20 h. The titers of phage of the positive tube and the negative control tube were determined respectively, using the same method as described above.

Based on the comparison result of phage titer, 5 mL of 2×YT liquid medium was added to the culture dish with the high phage titer after overnight culture, and all colonies were scraped off with a spreader rod. A sub-phage library was prepared according to the method in the above example. The above procedures were repeated until antibodies with the highest affinity for gp350 compared to the negative control were screened out.

For the antigen coating, blocking, incubation, washing, and elution processes, rotation speed was 15 rpm unless otherwise specified.

Antibodies with the highest affinity were detected using ELISA.

10 μL of the bacterial culture containing antibody with the highest affinity for gp350 was diluted with 1 mL of 2×YT liquid medium. Clonal streaking was performed on a 2×YT-GA solid culture plate and the plate was cultured overnight at 37° C. for 16-20 h. 192 single colonies were picked into a 96-well plate (each well containing 200 μL of 2×YT-GA liquid) and cultured statically at 37° C. until saturated. 2 μL of the saturated bacterial culture was transferred to a new 96-well plate (each well containing 200 μL of 2×YT-A liquid) to achieve an initial OD of approximately 0.03, and cultured statically at 37° C. for 2.5-3 h until OD reached approximately 0.5. 0.1 μL of the above helper phage was added to each well and infected at 37° C. for 30 min. 0.2 μL of Kanamycin was added to each well, and cultured at 30° C. overnight. Protein was used for coating (−/+) an ELISA plate overnight. The overnight-cultured 96-well plate was centrifuged at 4° C., 3400 g for 5 min. Liquid in the ELISA plate was discarded and the ELISA plate was washed once with 350 μL PBS. Blocking was performed with 350 μL of 3% BSA (in PBST) at 37° C. for 1 h. The blocking solution was discarded, the ELISA plate was washed once with 350 μL PBST, and tapped to remove liquid. 140 μL of 3% BSA (in PBST) was added to each well, then 60 μL of the above phage library expressing the antibody (antibody with the highest affinity, as the primary antibody) was added respectively and incubated at 37° C. for 1 h. The liquid (primary antibody) was discarded, the ELISA plate was washed for 5 times with 350 μL PBST, and tapped. 100 μL of M13 Antibody (RP) was added as the secondary antibody (1:8000 dilution in the blocking solution), incubated at 37° C. for 1 h. The liquid (secondary antibody) was discarded, the ELISA plate was washed for 5 times with 350 μL PBST, and tapped. 100 μL of TMB was added in the dark for 2-3 min, and the reaction was stopped with 100 μL of dilute hydrochloric acid (concentrated hydrochloric acid:water=1:12). The OD₄₅₀ and OD₆₃₀ of the ELISA plate were read. In the present example, the inventors ultimately obtained two antibodies with the best performance, designated as 1A12 and 2E4.

### Example 2: Expression and Purification of the Monoclonal Antibody 1A12

Full-length heavy chain fragment could be expressed by ligating upstream of the antibody heavy chain variable region to a CMV fragment, and downstream to the constant region of human IgG1 and a polyA fragment; and full-length light chain fragment could be expressed by ligating upstream of the antibody light chain variable region to a CMV fragment, and downstream to the constant region of the light chain κ/λ and a polyA fragment. Co-transfection of plasmids containing the above full-length sequences of the antibody heavy chain and light chain into 293T cells enabled antibody expression, and antibody purification was achieved using protein A beads.

The full-length heavy chain of 1A12 had 473 amino acid residues (excluding *), specifically as follows:

(SEQ ID NO: 13)
MGWSCIILFLVATATGVHSQVQLVQSGAEVKKPGASMKVSCKAS
GYTFAGYYIHWVRQAPGQGLEWMGSINPNSGKATYAQRFHGRVT
LTREMSINTAYMELTRLTSDDTAIYYCARDATYSTSFSPRWFDP
WGQGTLVTVSSASTKGPSVFPLAPSSKSTSGGTAALGCLVKDYF
PEPVTVSWNSGALTSGVHTFPAVLQSSGLYSLSSVVTVPSSSLG
TQTYICNVNHKPSNTKVDKRVEPKSCDKTHTCPPCPAPELLGGP
SVFLFPPKPKDTLMISRTPEVTCVVVDVSHEDPEVKFNWYVDGV
EVHNAKTKPREEQYNSTYRVVSVLTVLHQDWLNGKEYKCKVSNK
ALPAPIEKTISKAKGQPREPQVYTLPPSREEMTKNQVSLTCLVK
GFYPSDIAVEWESNGQPENNYKTTPPVLDSDGSFFLYSKLTVDK
SRWQQGNVFSCSVMHEALHNHYTQKSLSLSPGK*;

wherein, the underlined section in the sequence was the amino acid sequence of the heavy chain variable region (SEQ ID NO: 14). The underlined and bolded sections corresponded sequentially to the amino acid sequences of the three complementarity-determining regions CDR-H1 (SEQ ID NO: 15), CDR-H2 (SEQ ID NO: 16), and CDR-H3 (SEQ ID NO: 17) in the heavy chain variable region (IMGT definition scheme). The italicized section was the signal peptide. Amino acids 144-473 of SEQ ID NO: 13 constituted the heavy chain constant region. * indicated the stop codon.

CDR-H1, CDR-H2, and CDR-H3 in this heavy chain variable region defined by other CDR definition schemes were shown in Table 11.

TABLE 11
CDR-H1, CDR-H2, CDR-H3 in the Heavy Chain
Variable Region of Monoclonal Antibody 1A12
Defined by Other CDR Definition Schemes

| Other CDR | | | |
|---|---|---|---|
| definition | | | |
| schemes | CDR-H1 | CDR-H2 | CDR-H3 |
| Kabat | GYYIH | SINPNSGKA | DATYSTSF |
| | (SEQ ID | TYAQRFHG | SPRWFDP |
| | NO: 18) | (SEQ ID | (SEQ ID |
| | | NO: 19) | NO: 20) |
| Chothia | GYTFAGY | NPNSGK | DATYSTSF |
| | (SEQ ID | (SEQ ID | SPRWFDP |
| | NO: 21) | NO: 22) | (SEQ ID |
| | | | NO: 20) |
| Contact | AGYYIH | WMGSINPNS | ARDATYST |
| | (SEQ ID | GKAT | SFSPRWFD |
| | NO: 23) | (SEQ ID | (SEQ ID |
| | | NO: 24) | NO: 25) |

The coding gene for the full-length heavy chain of 1A12 had 1422 bases, specifically as follows:

(SEQ ID NO: 26)
5′-ATGGGATGGTCATGTATCATCCTTTTTCTAGTAGCAACTGCAA
CCGGTGTACATTCCCAGGTCCAGCTGGTACAGTCTGGGGCTGAGGT
GAAGAAGCCTGGGGCCTCAATGAAGGTCTCCTGCAAGGCTTCTGGG
TACACCTTCGCCGGCTACTATATACACTGGGTGCGACAGGCCCCTG
GACAAGGGCTTGAGTGGATGGGATCCATCAACCCTAATAGTGGTAA
AGCAACCTATGCACAGAGGTTTCACGGCAGGGTCACCTTGACCAGG
GAGATGTCCATTAATACAGCCTACATGGAACTGACCAGGCTGACAT
CTGACGACACGGCCATATACTACTGTGCGAGAGATGCGACATATAG
CACCAGTTTTTCCCCTCGTTGGTTCGACCCCTGGGGCCAGGGGACC
CTGGTCACCGTCTCCTCAGCGTCGACCAAGGGCCCATCGGTCTTCC
CCCTGGCACCCTCCTCCAAGAGCACCTCTGGGGGCACAGCGGCCCT
GGGCTGCCTGGTCAAGGACTACTTCCCCGAACCTGTGACGGTCTCG
TGGAACTCAGGCGCCCTGACCAGCGGCGTGCACACCTTCCCGGCTG
TCCTACAGTCCTCAGGACTCTACTCCCTCAGCAGCGTGGTGACCGT
GCCCTCCAGCAGCTTGGGCACCCAGACCTACATCTGCAACGTGAAT
CACAAGCCCAGCAACACCAAGGTGGACAAGAGAGTTGAGCCCAAAT
CTTGTGACAAAACTCACACATGCCCACCGTGCCCAGCACCTGAACT
CCTGGGGGGACCGTCAGTCTTCCTCTTCCCCCCAAAACCCAAGGAC
ACCCTCATGATCTCCCGGACCCCTGAGGTCACATGCGTGGTGGTGG
ACGTGAGCCACGAAGACCCTGAGGTCAAGTTCAACTGGTACGTGGA
CGGCGTGGAGGTGCATAATGCCAAGACAAAGCCGCGGGAGGAGCAG
TACAACAGCACGTACCGTGTGGTCAGCGTCCTCACCGTCCTGCACC
AGGACTGGCTGAATGGCAAGGAGTACAAGTGCAAGGTCTCCAACAA
AGCCCTCCCAGCCCCCATCGAGAAAACCATCTCCAAAGCCAAAGGG
CAGCCCCGAGAACCACAGGTGTACACCCTGCCCCCATCCCGGGAGG
AGATGACCAAGAACCAGGTCAGCCTGACCTGCCTGGTCAAAGGCTT
CTATCCCAGCGACATCGCCGTGGAGTGGGAGAGCAATGGGCAGCCG
GAGAACAACTACAAGACCACGCCTCCCGTGCTGGACTCCGACGGCT
CCTTCTTCCTCTATAGCAAGCTCACCGTGGACAAGAGCAGGTGGCA
GCAGGGGAACGTCTTCTCATGCTCCGTGATGCATGAGGCTCTGCAC
AACCACTACACGCAGAAGAGCCTCTCCCTGTCCCCGGGTAAAT
GA-3′.

Wherein, the underlined section in the sequence was the nucleotide sequence of the heavy chain variable region. The underlined and bolded sections corresponded sequentially to the nucleotide sequences of the three complementarity-determining regions CDR-H1, CDR-H2, and CDR-H3 in the heavy chain variable region (IMGT definition scheme). The first 3 bases at the 5′ end and the last 3 bases at the 3′ end were the start codon and stop codon, respectively. The italicized section was the signal peptide.

The full-length light chain of 1A12 had 233 amino acid residues (excluding *), specifically as follows:

(SEQ ID NO: 27)
MGWSCIILFLVATATGSWAEIVLTQSPGTLSLSPGERATLSCRASQ
SISSNSLAWYQQRPGQAPRLLIYGASSRATGIPDRFSGSGSGTDFT
LTISRLEPEDFAVYYCQQYGSSPRTFGPGTKVDIKGQPKAAPSVTL
FPPSSEELQANKATLVCLISDFYPGAVTVAWKADSSPVKAGVETTT
PSKQSNNKYAASSYLSLTPEQWKSHRSYSCQVTHEGSTVEKTVAPT
ECS*;

wherein, the underlined section in the sequence was the amino acid sequence of the light chain variable region (SEQ ID NO: 28). The underlined and bolded sections corresponded sequentially to the amino acid sequences of the three complementarity-determining regions CDR-L1 (SEQ ID NO: 29), CDR-L2 (GAS), and CDR-L3 (SEQ ID NO: 30) in the light chain variable region (IMGT definition scheme). The italicized section was the signal peptide. Amino acids 128-233 of SEQ ID NO: 27 constituted the light chain constant region. * indicated the stop codon.

CDR-L1, CDR-L2, and CDR-L3 in this light chain variable region defined by other CDR definition schemes were shown in Table 12.

TABLE 12
CDR-L1, CDR-L2, CDR-L3 in the Light Chain
Variable Region of Monoclonal Antibody 1A12
Defined by Other CDR Definition Schemes

| Other CDR | | | |
|---|---|---|---|
| definition | | | |
| schemes | CDR-L1 | CDR-L2 | CDR-L3 |
| Kabat | RASQSISSNSLA | GASSRAT | QQYGSSPRT |
| | (SEQ ID NO: 31) | (SEQ ID | (SEQ ID |
| | | NO: 32) | NO: 30) |
| Chothia | RASQSISSNSLA | GASSRAT | QQYGSSPRT |
| | (SEQ ID NO: 31) | (SEQ ID | (SEQ ID |
| | | NO: 32) | NO: 30) |
| Contact | SSNSLAWY | LLIYGAS | QQYGSSPR |
| | (SEQ ID NO: 33) | SRA | (SEQ ID |
| | | (SEQ ID | NO: 35) |
| | | NO: 34) | |

The coding gene for the full-length light chain of 1A12 had 702 bases, specifically as follows:

(SEQ ID NO: 36)
5′-ATGGGATGGTCATGTATCATCCTTTTTCTAGTAGCAACTGCAA
CCGGTTCCTGGGCCGAAATTGTGTTGACGCAGTCTCCAGGCACCCT
GTCTTTGTCTCCAGGGGAAAGAGCCACCCTCTCCTGCAGGGCCAGT
CAGAGTATTAGCAGCAACTCCTTGGCCTGGTACCAGCAGAGACCTG
GCCAGGCTCCCAGGCTCCTCATCTATGGTGCATCCAGCAGGGCCAC
TGGCATCCCAGACAGGTTCAGTGGCAGTGGGTCCGGGACAGACTTC
ACTCTCACCATCAGCAGACTGGAGCCTGAAGATTTTGCAGTGTATT
ACTGTCAGCAGTATGGTAGCTCACCTCGCACTTTCGGCCCTGGGAC
CAAAGTGGATATCAAAGGTCAGCCCAAGGCTGCCCCCTCGGTCACT
CTGTTCCCGCCCTCGAGTGAGGAGCTTCAAGCCAACAAGGCCACAC
TGGTGTGTCTCATAAGTGACTTCTACCCGGGAGCCGTGACAGTGGC
CTGGAAGGCAGATAGCAGCCCCGTCAAGGCGGGAGTGGAGACCACC
ACACCCTCCAAACAAAGCAACAACAAGTACGCGGCCAGCAGCTACC
TGAGCCTGACGCCTGAGCAGTGGAAGTCCCACAGAAGCTACAGCTG
CCAGGTCACGCATGAAGGGAGCACCGTGGAGAAGACAGTGGCCCCT
ACAGAATGTTCATAG-3′.

Wherein, the underlined section in the sequence was the nucleotide sequence of the light chain variable region. The underlined and bolded sections corresponded sequentially to the nucleotide sequences of the three complementarity-determining regions CDR-L1, CDR-L2, and CDR-L3 in the light chain variable region. The first 3 bases at the 5′ end and the last 3 bases at the 3′ end were the start codon and stop codon, respectively. The italicized section was the signal peptide.

### Example 3: Expression and Purification of the Monoclonal Antibody 2E4

Full-length heavy chain fragment could be expressed by ligating upstream of the antibody heavy chain variable region to a CMV fragment, and downstream to the constant region of human IgG1 and a polyA fragment; and full-length light chain fragment could be expressed by ligating upstream of the antibody light chain variable region to a CMV fragment, and downstream to the constant region of the light chain κ/λ and a polyA fragment. Co-transfection of plasmids containing the above full-length sequences of the antibody heavy chain and light chain into 293T cells enabled antibody expression, and antibody purification was achieved using protein A beads.

The full-length heavy chain of 2E4 had 468 amino acid residues (excluding *), specifically as follows:

(SEQ ID NO: 37)
MGWSCIILFLVATATGVHSQVQLVQSGAEVKKPGASVKVSCRASGY
TFTSHYIHWVRQAPGQGLEWMGITNPSTGSTNYAQQFQGRVTMTRD
TSTSTVYMEVSSLRSEDTAIYYCARDVKRLGNLDYWGQGTLVTVSS
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSNT
KVDKRVEPKSCDKTHTCPPCPAPELLGGPSVFLFPPKPKDTLMISR
TPEVTCVVVDVSHEDPEVKFNWYVDGVEVHNAKTKPREEQYNSTYR
VVSVLTVLHQDWLNGKEYKCKVSNKALPAPIEKTISKAKGQPREPQ
VYTLPPSREEMTKNQVSLTCLVKGFYPSDIAVEWESNGQPENNYKT
TPPVLDSDGSFFLYSKLTVDKSRWQQGNVFSCSVMHEALHNHYTQK
SLSLSPGK*.

Wherein, the underlined section in the sequence was the amino acid sequence of the heavy chain variable region (SEQ ID NO: 38). The underlined and bolded sections corresponded sequentially to the amino acid sequences of the three complementarity-determining regions CDR-H4 (SEQ ID NO: 39), CDR-H5 (SEQ ID NO: 40), and CDR-H6 (SEQ ID NO: 41) in the heavy chain variable region (IMGT definition scheme). The italicized section was the signal peptide. Amino acids 139-468 of SEQ ID NO: 37 constituted the heavy chain constant region. * indicated the stop codon.

CDR-H4, CDR-H5, and CDR-H6 in this heavy chain variable region defined by other CDR definition schemes were shown in Table 13.

TABLE 13
CDR-H4, CDR-H5, CDR-H6 in the Heavy Chain
Variable Region of Monoclonal Antibody 2E4
Defined by Other CDR Definition Schemes

| Other CDR | | | |
|---|---|---|---|
| definition | | | |
| schemes | CDR-H4 | CDR-H5 | CDR-H6 |
| Kabat | SHYIH | ITNPSTGSTNYAQQ | DVKRLGNLDY |
| | (SEQ ID | FQG | (SEQ ID |
| | NO: 42) | (SEQ ID NO: 43) | NO: 44) |
| Chothia | GYTFTSH | NPSTGS | DVKRLGNLDY |
| | (SEQ ID | (SEQ ID NO: 46) | (SEQ ID |
| | NO: 45) | | NO: 44) |
| Contact | TSHYIH | WMGITNPSTGSTN | ARDVKRLGNLD |
| | (SEQ ID | (SEQ ID NO: 48) | (SEQ ID |
| | NO: 47) | | NO: 49) |

The coding gene for the full-length heavy chain of 2E4 had 1408 bases, specifically as follows:

(SEQ ID NO: 50)
5′-ATGGGATGGTCATGTATCATCCTTTTTCTAGTAGCAACTGCAAC
CGGTGTACATTCCCAGGTCCAGCTGGTGCAGTCTGGGGCTGAGGTGA
AGAAGCCTGGGGCCTCAGTGAAGGTGTCCTGCAGGGCATCTGGATAC
ACTTTCACCAGTCACTATATTCACTGGGTGCGACAGGCCCCTGGCCA
AGGGCTTGAGTGGATGGGAATAACTAATCCTAGTACTGGTTCCACAA
ACTACGCACAGCAGTTCCAGGGCAGAGTCACCATGACCAGGGACACG
TCCACGAGCACAGTCTACATGGAGGTGAGCAGCCTGAGATCTGAGGA
CACGGCGATATATTACTGTGCGAGAGACGTGAAGCGACTGGGGAACC
TTGACTACTGGGGCCAGGGAACCCTGGTCACCGTCTCCTCAGCGTCG
ACCAAGGGCCCATCGGTCTTCCCCCTGGCACCCTCCTCCAAGAGCAC
CTCTGGGGGCACAGCGGCCCTGGGCTGCCTGGTCAAGGACTACTTCC
CCGAACCTGTGACGGTCTCGTGGAACTCAGGCGCCCTGACCAGCGGC
GTGCACACCTTCCCGGCTGTCCTACAGTCCTCAGGACTCTACTCCCT
CAGCAGCGTGGTGACCGTGCCCTCCAGCAGCTTGGGCACCCAGACCT
ACATCTGCAACGTGAATCACAAGCCCAGCAACACCAAGGTGGACAAG
AGAGTTGAGCCCAAATCTTGTGACAAAACTCACACATGCCCACCGTG
CCCAGCACCTGAACTCCTGGGGGGACCGTCAGTCTTCCTCTTCCCCC
CAAAACCCAAGGACACCCTCATGATCTCCCGGACCCCTGAGGTCACA
TGCGTGGTGGTGGACGTGAGCCACGAAGACCCTGAGGTCAAGTTCAA
CTGGTACGTGGACGGCGTGGAGGTGCATAATGCCAAGACAAAGCCGC
GGGAGGAGCAGTACAACAGCACGTACCGTGTGGTCAGCGTCCTCACC
GTCCTGCACCAGGACTGGCTGAATGGCAAGGAGTACAAGTGCAAGGT
CTCCAACAAAGCCCTCCCAGCCCCCATCGAGAAAACCATCTCCAAAG
CCAAAGGGCAGCCCCGAGAACCACAGGTGTACACCCTGCCCCCATCC
CGGGAGGAGATGACCAAGAACCAGGTCAGCCTGACCTGCCTGGTCAA
AGGCTTCTATCCCAGCGACATCGCCGTGGAGTGGGAGAGCAATGGGC
AGCCGGAGAACAACTACAAGACCACGCCTCCCGTGCTGGACTCCGAC
GGCTCCTTCTTCCTCTATAGCAAGCTCACCGTGGACAAGAGCAGGTG
GCAGCAGGGGAACGTCTTCTCATGCTCCGTGATGCATGAGGCTCTGC
ACAACCACTACACGCAGAAGAGCCTCTCCCTGTCCCCGGGTAAATG
A-3′.

Wherein, the underlined section in the sequence was the nucleotide sequence of the heavy chain variable region. The underlined and bolded sections corresponded sequentially to the 25 nucleotide sequences of the three complementarity-determining regions CDR-H4, CDR-H5, and CDR-H6 in the heavy chain variable region (IMGT definition scheme). The first 3 bases at the 5′ end and the last 3 bases at the 3′ end were the start codon and stop codon, respectively. The italicized section was the signal peptide.

The full-length light chain of 2E4 had 235 amino acid residues (excluding *), specifically as follows:

(SEQ ID NO: 51)
MGWSCIILFLVATATGSWAQPVLTQPPSASGTPGQRVTISCSGSSSN
IGRNYVYWYQQFPGTAPKLLIYRNNQRPSGVPDRFSASKSGTSASLA
ISGLRSEDEADYYCAAWDDRLSGAVFGGGTQLTVLGQPKAAPSVTLF
PPSSEELQANKATLVCLISDFYPGAVTVAWKADSSPVKAGVETTTPS
KQSNNKYAASSYLSLTPEQWKSHRSYSCQVTHEGSTVEKTVAPTEC
S*.

Wherein, the underlined section in the sequence was the amino acid sequence of the light chain variable region (SEQ ID NO: 52). The underlined and bolded sections corresponded sequentially to the amino acid sequences of the three complementarity-determining regions CDR-L4 (SEQ ID NO: 53), CDR-L5 (RNN), and CDR-L6 (SEQ ID NO: 54) in the light chain variable region (IMGT definition scheme). The italicized section was the signal peptide. Amino acids 130-235 of SEQ ID NO: 51 constituted the light chain constant region. * indicated the stop codon.

CDR-L4, CDR-L5, and CDR-L6 in this light chain variable region defined by other CDR definition schemes were shown in Table 14.

TABLE 14
CDR-L4, CDR-L5, CDR-L6 in the Light Chain
Variable Region of Monoclonal Antibody 2E4
Defined by Other CDR Definition Schemes

| Other CDR | | | |
|---|---|---|---|
| definition | | | |
| schemes | CDR-L4 | CDR-L5 | CDR-L6 |
| Kabat | SGSSSNIGR | RNNQRPS | AAWDDRLSGAV |
| | NYVY | (SEQ ID | (SEQ ID |
| | (SEQ ID | NO: 56) | NO: 54) |
| | NO: 55) | | |
| Chothia | SGSSSNIGR | RNNQRPS | AAWDDRLSGAV |
| | NYVY | (SEQ ID | (SEQ ID |
| | (SEQ ID | NO: 56) | NO: 54) |
| | NO: 55) | | |
| Contact | IGRNYVYWY | LLIYRNNQRP | AAWDDRLSGA |
| | (SEQ ID | (SEQ ID | (SEQ ID |
| | NO: 57) | NO: 58) | NO: 59) |

The coding gene for the full-length light chain of 2E4 had 708 bases, specifically as follows:

(SEQ ID NO: 60)
5′-ATGGGATGGTCATGTATCATCCTTTTTCTAGTAGCAACTGC
AACCGGTTCCTGGGCCCAGCCAGTGCTGACTCAGCCACCCTCAG
CGTCTGGGACCCCCGGGCAGAGGGTCACCATTTCTTGTTCTGGA
AGCAGCTCCAACATCGGAAGGAATTATGTATACTGGTATCAGCA
GTTCCCAGGAACGGCCCCCAAACTCCTCATCTATAGGAATAATC
AGCGGCCCTCAGGGGTCCCTGACCGATTCTCTGCCTCCAAGTCT
GGTACCTCAGCCTCCCTGGCCATCAGTGGGCTCCGGTCCGAGGA
TGAGGCTGATTATTATTGTGCAGCATGGGATGACAGATTGAGTG
GCGCTGTGTTCGGAGGAGGCACCCAGCTGACCGTCCTCGGTCAG
CCCAAGGCTGCCCCCTCGGTCACTCTGTTCCCGCCCTCGAGTGA
GGAGCTTCAAGCCAACAAGGCCACACTGGTGTGTCTCATAAGTG
ACTTCTACCCGGGAGCCGTGACAGTGGCCTGGAAGGCAGATAGC
AGCCCCGTCAAGGCGGGAGTGGAGACCACCACACCCTCCAAACA
AAGCAACAACAAGTACGCGGCCAGCAGCTACCTGAGCCTGACGC
CTGAGCAGTGGAAGTCCCACAGAAGCTACAGCTGCCAGGTCACG
CATGAAGGGAGCACCGTGGAGAAGACAGTGGCCCCTACAGAATG
TTCATAG-3′.

Wherein, the underlined section in the sequence was the nucleotide sequence of the light chain variable region. The underlined and bolded sections corresponded sequentially to the nucleotide sequences of the three complementarity-determining regions CDR-L4, CDR-L5, and CDR-L6 in the light chain variable region. The first 3 bases at the 5′ end and the last 3 bases at the 3′ end were the start codon and stop codon, respectively. The italicized section was the signal peptide.

### Efficacy Example 1: Efficacy of Monoclonal Antibody 1A12

### 1. Affinity of Antibody 1A12 Determined by ELISA.

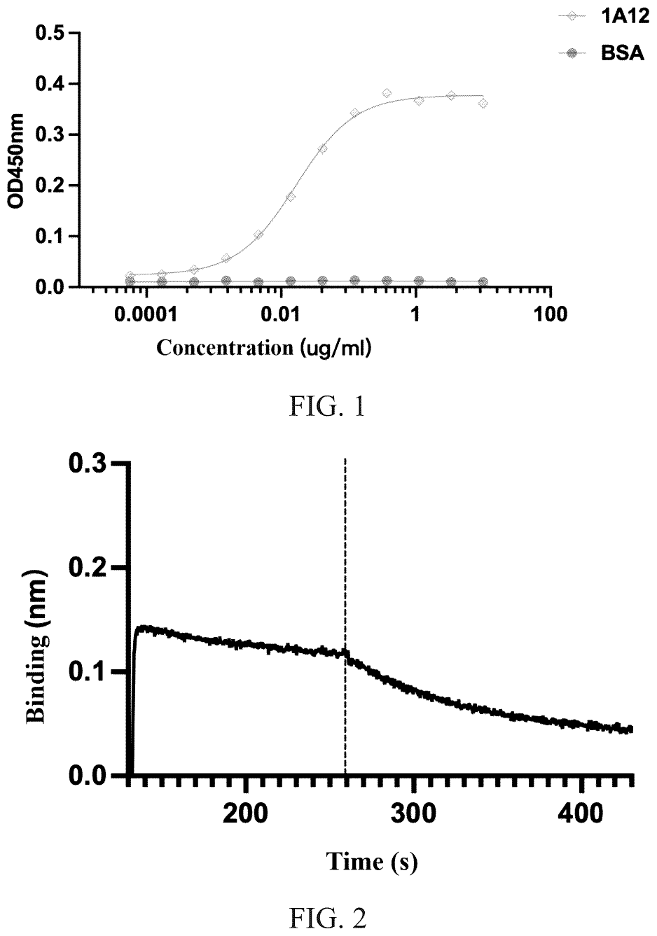

To determine the affinity of antibody 1A12, the ELISA method was used in the present efficacy example. First of all, the gp350 protein was diluted with PBS buffer, added to an ELISA plate with 100 ng per well, and incubated overnight at 4° C. Unbound antigen was discarded the next day, and the plate was washed 3 times with PBST buffer, 3 min each time. Then, 5% BSA solution was added for blocking, incubated at 37° C. for 1-2 h, and the plate was washed with PBST again. Next, the antibody 1A12 was diluted in gradients, added to the corresponding wells, and incubated at 37° C. for 1-2 h to promote antibody binding to the coated gp350 protein (BSA solution served as a negative control). After incubation, the plate was washed 3 times with PBST, then HRP-labeled enzyme-conjugated secondary antibody (e.g., anti-human IgG secondary antibody) was added and incubated at 37° C. for 1 h. Subsequently, the plate was washed 3 times with PBST, TMB chromogenic substrate was added, and incubated at room temperature for 5-15 min. When the color reached desired intensity, the reaction was stopped with dilute hydrochloric acid. Finally, the absorbance (OD value) of each well was measured at 450 nm wavelength. The curve of antibody concentration versus OD value was plotted and the result was shown in FIG. 1. As can be seen, IC₅₀ of the 1A12 antibody is 0.017 g/mL, indicating that the 1A12 antibody has high affinity for the gp350 antigen.

### 2. Affinity of Antibody 1A12 Determined by Bio-Layer Interferometry (BLI).

BLI was performed according to conventional method in the art. In the present example, the specific procedures were as follows: a biosensor (Sartorius Octet® SA probe, Sartorius) was immersed in a buffer (a mixture of KB buffer, 0.1% BSA, and 0.02% Tween 20) for equilibration; the biosensor was then removed and immersed in a solution containing 5 g/mL gp350-Biotin (biotin-labeled gp350 protein), during which the gp350 antigen in the solution would bind to the Streptavidin (SA) bio-probe surface, causing an increase in layer thickness; the biosensor with the immobilized antigen of known concentration was then immersed in buffer to establish a baseline; the biosensor with the immobilized antigen of known concentration was immersed in a sample solution containing the 1A12 antibody for about 100 seconds, wherein specific binding between the antigen and the antibody would cause an increase in layer thickness; the biosensor bound with the 1A12 antibody was then immersed in buffer for dissociation, during which the test antibody (1A12 antibody) would detach from the biosensor surface, causing a decrease in layer thickness; and by real-time monitoring of the biolayer thickness of the biosensor during the experiment, the kinetic constant of the test sample (1A12 antibody) was obtained. The result was shown in FIG. 2. As can be seen, KD(M) of the 1A12 antibody is 3.415E⁻¹⁰, indicating that the 1A12 antibody has high affinity for the gp350 antigen.

### 3. Neutralizing Activity of Antibody 1A12

### (1) Preparation of EBV Virus:

1) CNE2 cells infected with EBV-GFP (the virus has been disclosed in the literature: An Antibody Targeting the Fusion Machinery Neutralizes Dual-Tropic Infection and Defines a Site of Vulnerability on Epstein-Barr Virus) were cultured in RPMI1640+5% FBS medium in a 37° C. incubator (5% CO₂). When the cells reached 90% density (in a 10 cm dish), TPA to a final concentration of 20 ng/mL and sodium butyrate (NaB) to a final concentration of 2.5 mM were added for induction. The medium was changed after 12 hours.
2) 48-72 hours after the change of medium, the culture supernatant was harvested for virus isolation and purification. The supernatant was directly aspirated, centrifuged, filtered through a 0.45 m filter, concentrated and resuspended with serum-free RPMI1640. The resuspended virus was either used immediately for infection or stored at −80° C.

### (2) Detection of B Cell Neutralizing Activity of 1A12 Monoclonal Antibody

1) The 1A12 monoclonal antibody from the above example was adjusted to a concentration of 2 mg/mL. 60 μL of RPMI1640 medium was added to each well of a new 96-well plate. 90 μL of 100 g/mL 1A12 antibody diluted with RPMI1640 was added to the first well (the first well did not contain RPMI1640 medium).
2) 3-fold gradient dilutions were performed (30 μL diluted 1A12 antibody was aspirated from the first well and added to the second well, and so on; 30 μL from the last well was discarded after mixing, resulting in a final volume of 60 μL per well). Then, 60 μL of virus dilution (virus was diluted with DMEM medium to a titer of approximately 4×10⁶/mL) was added to each well, incubated at 37° C. for 2 hours, and then 1×10⁶ Raji cells were added to each well. The plate was placed in a 37° C. incubator and cultured for 48 hours before detection.
3) The Raji cells were aspirated to prepare a cell suspension. The infection rate was detected by flow cytometry. The inhibition rate (neutralization efficiency, %) of the antibody in the Raji B-cell infection model was calculated by detecting the reduction percentage in the number of GFP-positive cells in the antibody-treated group compared to the infection control group (added with an equal volume of RPMI 1640). IC₅₀ of the 1A12 monoclonal antibody was calculated and plotted using Prism. Antibody 2G4 (2G4 was an anti-Ebola virus antibody, disclosed in the literature: Audet, J. et al. Molecular characterization of the monoclonal antibodies composing ZMAb: a protective cocktail against Ebola virus. Sci. Rep. 4, 6881(2014).) was used as a negative control antibody.

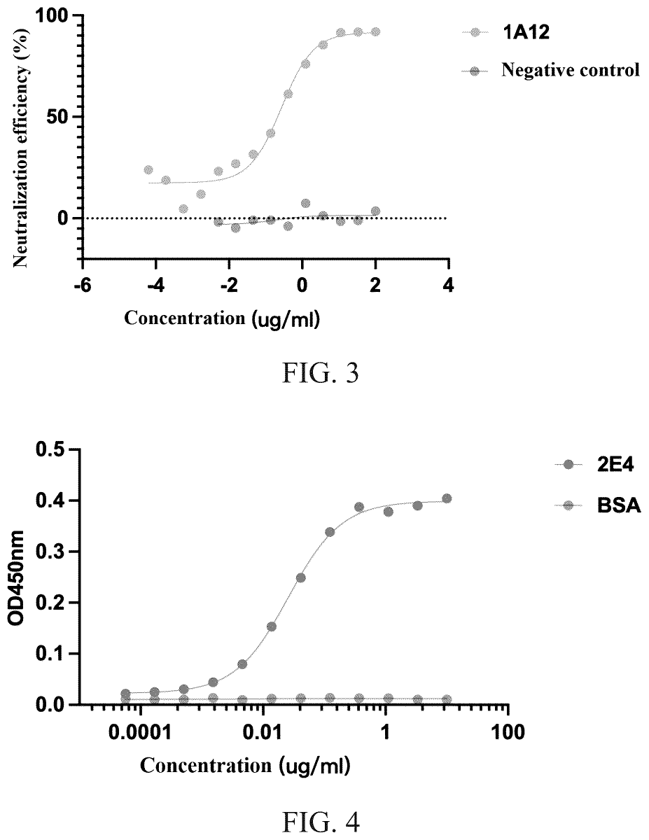

The result was shown in FIG. 3. As can be seen, IC₅₀ of monoclonal antibody 1A12 in the B cell infection model is 0.27 g/mL, while the control antibody shows no neutralizing activity; and monoclonal antibody 1A12 can significantly inhibit EBV infection of B cells.

### Efficacy Example 2: Efficacy of Monoclonal Antibody 2E4

### 1. Affinity of Antibody 2E4 Determined by ELISA.

The experimental method was identical to that in Efficacy Example 1.

The result was shown in FIG. 4. As can be seen, IC₅₀ of the 2E4 antibody is 0.026 g/mL, indicating that the 2E4 antibody has high affinity for the gp350 antigen.

### 2. Affinity of Antibody 2E4 Determined by Bio-Layer Interferometry (BLI).

The experimental method was identical to that in Efficacy Example 1.

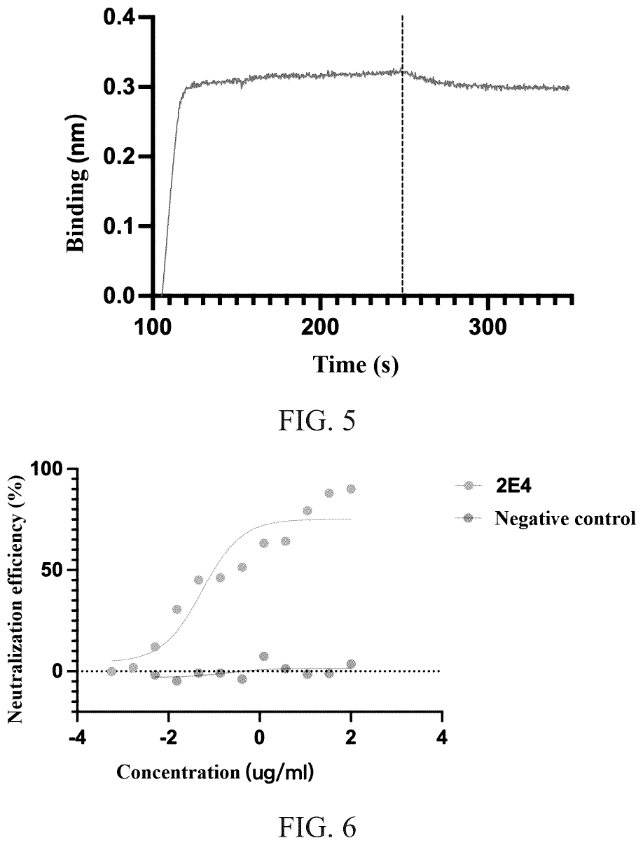

The result was shown in FIG. 5. As can be seen, KD(M) of the 2E4 antibody is 4.851E⁻⁹; indicating that the 2E4 antibody has high affinity for the gp350 antigen.

### 3. Neutralizing Activity of Antibody 2E4

### Preparation of EBV Virus:

Identical to that in Efficacy Example 1.

### 4. Detection of B Cell Neutralizing Activity of 2E4 Monoclonal Antibody:

1) The 2E4 monoclonal antibody from the above example was adjusted to a concentration of 2 mg/mL. 60 μL of RPMI1640 medium was added to each well of a new 96-well plate. 90 μL of 100 g/mL 2E4 antibody diluted with RPMI1640 was added to the first well (the first well did not contain RPMI1640 medium).
2) 3-fold gradient dilutions were performed (30 μL diluted 2E4 antibody was aspirated from the first well and added to the second well, and so on; 30 μL from the last well was discarded after mixing, resulting in a final volume of 60 μL per well). Then, 60 μL of virus dilution (virus was diluted with DMEM medium to a titer of approximately 4×10⁶/mL) was added to each well, incubated at 37° C. for 2 hours, and then 1×10⁶ Raji cells were added to each well. The plate was placed in a 37° C. incubator and cultured for 48 hours before detection.
3) The Raji cells were aspirated to prepare a cell suspension. The infection rate was detected by flow cytometry. The inhibition rate (neutralization efficiency, %) of the antibody in the Raji B-cell infection model was calculated by detecting the reduction percentage in the number of GFP-positive cells in the antibody-treated group compared to the infection control group (added with an equal volume of RPMI 1640). IC₅₀ of the 2E4 monoclonal antibody was calculated and plotted using Prism. Antibody 2G4 was used as a negative control antibody.

The result was shown in FIG. 6. As can be seen, IC₅₀ of monoclonal antibody 2E4 in the B cell infection model is 0.054 g/mL, while the control antibody shows no neutralizing activity; and Monoclonal antibody 2E4 can significantly inhibit EBV infection of B cells.

1. A monoclonal antibody against EBV gp350 protein or an antigen-binding fragment thereof, wherein the monoclonal antibody or the antigen-binding fragment thereof comprises a heavy chain and a light chain;
the monoclonal antibody comprises any one of A) or B):
A)
the heavy chain comprises:
a heavy chain variable region comprising CDR-H1, CDR-H2, and CDR-H3 of the heavy chain variable region, wherein the heavy chain variable region has an amino acid sequence shown in SEQ ID NO: 14;
the light chain comprises:
a light chain variable region comprising CDR-L1, CDR-L2, and CDR-L3 of the light chain variable region, wherein the light chain variable region has an amino acid sequence shown in SEQ ID NO: 28;
B)
the heavy chain comprises:
a heavy chain variable region comprising CDR-H4, CDR-H5, and CDR-H6 of the heavy chain variable region, wherein the heavy chain variable region has an amino acid sequence shown in SEQ ID NO: 38;
the light chain comprises:
a light chain variable region comprising CDR-L4, CDR-L5, and CDR-L6 of the light chain variable region, wherein the light chain variable region has an amino acid sequence shown in SEQ ID NO: 52.

2. The monoclonal antibody or the antigen-binding fragment thereof according to claim 1, wherein:
A)
amino acid sequences of CDR-H1, CDR-H2, CDR-H3, CDR-L1, CDR-L2, and CDR-L3 are as shown in SEQ ID NO: 18, SEQ ID NO: 19, SEQ ID NO: 20, SEQ ID NO: 31, SEQ ID NO: 32, and SEQ ID NO: 30, respectively, wherein the CDRs are defined according to the Kabat definition scheme; or
the amino acid sequences of CDR-H1, CDR-H2, CDR-H3, CDR-L1, and CDR-L3 are as shown in SEQ ID NO: 15, SEQ ID NO: 16, SEQ ID NO: 17, SEQ ID NO: 29, and SEQ ID NO:
30, respectively, and the amino acid sequence of CDR-L2 is: GAS, wherein the CDRs are defined according to the IMGT definition scheme; or
the amino acid sequences of CDR-H1, CDR-H2, CDR-H3, CDR-L1, CDR-L2, and CDR-L3 are as shown in SEQ ID NO: 21, SEQ ID NO: 22, SEQ ID NO: 20, SEQ ID NO: 31, SEQ ID NO: 32, and SEQ ID NO: 30, respectively, wherein the CDRs are defined according to the Chothia definition scheme; or
the amino acid sequences of CDR-H1, CDR-H2, CDR-H3, CDR-L1, CDR-L2, and CDR-L3 are as shown in SEQ ID NO: 23, SEQ ID NO: 24, SEQ ID NO: 25, SEQ ID NO: 33, SEQ ID NO: 34, and SEQ ID NO: 35, respectively, wherein the CDRs are defined according to the Contact definition scheme;
B)
amino acid sequences of CDR-H4, CDR-H5, CDR-H6, CDR-L4, CDR-L5, and CDR-L6 are as shown in SEQ ID NO: 42, SEQ ID NO: 43, SEQ ID NO: 44, SEQ ID NO: 55, SEQ ID NO: 56, and SEQ ID NO: 54, respectively, wherein the CDRs are defined according to the Kabat definition scheme; or
the amino acid sequences of CDR-H4, CDR-H5, CDR-H6, CDR-L4, and CDR-L6 are as shown in SEQ ID NO: 39, SEQ ID NO: 40, SEQ ID NO: 41, SEQ ID NO: 53, and SEQ ID NO: 54, respectively, and the amino acid sequence of CDR-L5 is: RNN, wherein the CDRs are defined according to the IMGT definition scheme; or
the amino acid sequences of CDR-H4, CDR-H5, CDR-H6, CDR-L4, CDR-L5, and CDR-L6 are as shown in SEQ ID NO: 45, SEQ ID NO: 46, SEQ ID NO: 44, SEQ ID NO: 55, SEQ ID NO: 56, and SEQ ID NO: 54, respectively, wherein the CDRs are defined according to the Chothia definition scheme; or
the amino acid sequences of CDR-H4, CDR-H5, CDR-H6, CDR-L4, CDR-L5, and CDR-L6 are as shown in SEQ ID NO: 47, SEQ ID NO: 48, SEQ ID NO: 49, SEQ ID NO: 57, SEQ ID NO: 58, and SEQ ID NO: 59, respectively, wherein the CDRs are defined according to the Contact definition scheme.

3. The monoclonal antibody or the antigen-binding fragment thereof according to claim 1, wherein the monoclonal antibody or the antigen-binding fragment thereof comprises at least one of a full-length antibody, Fab, Fab′, F(ab′)2, Fv, scFv, a bispecific antibody, or a multispecific antibody.

4. A recombinant protein comprising the monoclonal antibody or the antigen-binding fragment thereof according to claim 1; and
optionally a tag sequence facilitating expression and/or purification.

5. A biological material related to the monoclonal antibody or the antigen-binding fragment thereof according to claim 1, wherein the biological material comprises at least one of h1) to h16):
h1) a nucleic acid molecule encoding the monoclonal antibody or the antigen-binding fragment thereof;
h2) an expression cassette comprising the nucleic acid molecule of h1);
h3) a vector comprising the nucleic acid molecule of h1);
h4) a vector comprising the expression cassette of h2);
h5) a transgenic cell line comprising the nucleic acid molecule of h1);
h6) a transgenic cell line comprising the expression cassette of h2);
h7) a transgenic cell line comprising the vector of h3);
h8) a transgenic cell line comprising the vector of h4);
h9) a microorganism comprising the nucleic acid molecule of h1);
h10) a microorganism comprising the expression cassette of h2);
h11) a microorganism comprising the vector of h3);
h12) a microorganism comprising the vector of h4);
h13) a virus comprising the nucleic acid molecule of h1);
h14) a virus comprising the expression cassette of h2);
h15) a virus comprising the vector of h3); or
h16) a virus comprising the vector of h4).

6. A conjugate comprising the monoclonal antibody or the antigen-binding fragment thereof according to claim 1;
and a conjugated moiety, wherein the conjugated moiety comprises at least one of a detectable label, a drug, a toxin, a cytokine, a radionuclide, or an enzyme.

7. A drug comprising the monoclonal antibody or the antigen-binding fragment thereof according to claim 1.

8. The drug according to claim 7, wherein the drug has at least one function of i1) to i2):
i1) preventing EBV infection; or
i2) treating and/or preventing diseases caused by EBV infection.

9. A product comprising the monoclonal antibody or the antigen-binding fragment thereof according to claim 1,
wherein the product comprises at least one of a reagent, an assay plate, a kit, or a detection chip.

10. The product according to claim 9, wherein the product has at least one function of j1) to j3):
j1) detecting the presence or level of gp350 protein in a sample;
j2) detecting EBV; or
j3) diagnosing diseases caused by EBV infection.

11. A pharmaceutical composition comprising the monoclonal antibody or the antigen-binding fragment thereof according to claim 1, wherein the pharmaceutical composition further comprises a pharmaceutically acceptable carrier.

12. A vaccine, comprising the monoclonal antibody or the antigen-binding fragment thereof according to claim 1, and an adjuvant.

13. A biological material related to the recombinant protein according to claim 4, wherein the biological material comprises at least one of h1) to h16):
h1) a nucleic acid molecule encoding the recombinant protein;
h2) an expression cassette comprising the nucleic acid molecule of h1);
h3) a vector comprising the nucleic acid molecule of h1);
h4) a vector comprising the expression cassette of h2);
h5) a transgenic cell line comprising the nucleic acid molecule of h1);
h6) a transgenic cell line comprising the expression cassette of h2);
h7) a transgenic cell line comprising the vector of h3);
h8) a transgenic cell line comprising the vector of h4);
h9) a microorganism comprising the nucleic acid molecule of h1);
h10) a microorganism comprising the expression cassette of h2);
h11) a microorganism comprising the vector of h3);
h12) a microorganism comprising the vector of h4);
h13) a virus comprising the nucleic acid molecule of h1);
h14) a virus comprising the expression cassette of h2);
h15) a virus comprising the vector of h3); or
h16) a virus comprising the vector of h4).

14. A conjugate comprising the recombinant protein according to claim 4,
and a conjugated moiety, wherein the conjugated moiety comprises at least one of a detectable label, a drug, a toxin, a cytokine, a radionuclide, or an enzyme.

15. A drug comprising the recombinant protein according to claim 4, wherein the drug has at least one function of i1) to i2):
i1) preventing EBV infection; or
i2) treating and/or preventing diseases caused by EBV infection.

16. A product comprising the recombinant protein according to claim 4, wherein the product comprises at least one of a reagent, an assay plate, a kit, or a detection chip, and the product has at least one function of j1) to j3):
j1) detecting the presence or level of gp350 protein in a sample;
j2) detecting EBV; or
j3) diagnosing diseases caused by EBV infection.

17. A drug comprising the conjugate according to claim 6, wherein the drug has at least one function of i1) to i2):
i1) preventing EBV infection; or
i2) treating and/or preventing diseases caused by EBV infection.

18. A product comprising the conjugate according to claim 6, wherein the product comprises at least one of a reagent, an assay plate, a kit, or a detection chip, and the product has at least one function of j1) to j3):
j1) detecting the presence or level of gp350 protein in a sample;
j2) detecting EBV; or
j3) diagnosing diseases caused by EBV infection.

19. A vaccine, comprising the recombinant protein according to claim 4, and an adjuvant.

20. A vaccine, comprising the biological material according to claim 5, and an adjuvant.


SEQUENCE LISTING (61 sequences)

SEQ ID NO: 1 - DNA, 2601 bp, synthetic construct
  source 1..2601: mol_type = other DNA
gccaccatggacgccatgaagaggggcctgtgctgcgtgctgctgctgtgtggcgccgtgtttgtgtcccccagccaggaaatccacgcccggttcagaagaggcgccagaggatcccaccaccatcatcaccatcatcaccaccatgaggcagccttgcttgtgtgtcagtacaccatccagagcctgatccatctcacgggtgaagatcctggttttttcaatgttgagattccggaattcccattttaccccacatgcaatgtttgtacggcagatgtcaatgtaactatcaatttcgatgtcgggggcaaaaagcatcaacttgatcttgactttggccagctgacaccccatacgaaggctgtctaccaacctcgaggtgcatttggtggctcagaaaatgccaccaatctctttctactggagctccttggtgcaggagaattggctctaactatgcggtctaagaagcttccaattaacgtcaccaccggagaggagcaacaagtaagcctggaatctgtagatgtctactttcaagatgtgtttggaaccatgtggtgccaccatgcagaaatgcaaaaccccgtgtacctgataccagaaacagtgccatacataaagtgggataactgtaattctaccaatataacggcagtagtgagggcacaggggctggatgtcacgttacccttaagtttgccaacgtcagctcaagactcgaatttcagcgtaaaaacacaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatcctgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaaaacactcattatcacacgaacggctaccaatgccaccacaacaacccacaaggttatattctccaaggcacccgagagcaccaccacctcccctaccttgaatacaactggatttgctgatcccaatacaacgacaggtctacccagctctactcacgtgcctaccaacctcaccgcacctgcaagcacaggccccactgtatccacagcggatgtcaccagcccaacaccagccggcacaacgtcaggcgcatcaccggtgacaccaagtccatctccacgggacaacggcacagaaagtaaggcccccgacatgaccagccccacatcagcagtgactaccccaaccccaaatggcaccagccccaccccagcaatgactaccccaaccccaaatgccaccagccccaccttgggaaaaacaagtcccacctcagcagtgactaccccaaccccaaatgccaccagccccaccccagcagtgactaccccaaccccaaatgccaccagccctactgtgggagaaacaagtccacaggcaaatgccaccaaccacaccttaggaggaacaagtcccaccccagtagttaccagcccaccaaaaaatgcaaccagtgatgttaccacaggccaacataacagaacttcaagttcaacctcttccatgtcactgagacccagttcaatcccagagacaacgtcacatatgcctttactaacctccgctcacccaacaggtggtgaaaatataacacaggtgacaccagcctctatcagcacacatcatgtgtccaccagttcgccagcgccccgcccaggcaccaccagccaagcgtcaggccctggaaacagttccacatccacaaaaccgggggaggttaatgtcaccaaaggcacgccccccaaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcgatgattcaactacgccaagaccgagatacaatgcgaccacctatctacctcccagcacttctagcaaactgcggccccgctggacttttacgagcccaccggttaccacagcccaagccaccgtgcctgtcccgccaacgtcccagcccagattctcaaacctccaccaccatcatcaccatcatcaccaccattaa

SEQ ID NO: 2 - DNA, 20 bp, synthetic construct
  source 1..20: mol_type = other DNA
gaggcagccttgcttgtgtg

SEQ ID NO: 3 - DNA, 22 bp, synthetic construct
  source 1..22: mol_type = other DNA
gaggtttgagaatctgggctgg

SEQ ID NO: 4 - protein, 827 aa, synthetic construct
HHHHHHHHHHEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTQMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPRDNGTESKAPDMTSPTSAVTTPTPNGTSPTPAMTTPTPNATSPTLGKTSPTSAVTTPTPNATSPTPAVTTPTPNATSPTVGETSPQANATNHTLGGTSPTPVVTSPPKNATSDVTTGQHNRTSSSTSSMSLRPSSIPETTSHMPLLTSAHPTGGENITQVTPASISTHHVSTSSPAPRPGTTSQASGPGNSSTSTKPGEVNVTKGTPPKNATSPQAPSGQKTAVPTVTSTGGKANSTTGGKHTTGHGARTSTEPTTDYGDDSTTPRPRYNATTYLPPSTSSKLRPRWTFTSPPVTTAQATVPVPPTSQPRFSNLHHHHHHHHHH

SEQ ID NO: 5 - DNA, 53 bp, synthetic construct
  source 1..53: mol_type = other DNA
cctttctatgcggcccagccggccgagctccagtctgtsbtgacgcagccgcc

SEQ ID NO: 6 - DNA, 41 bp, synthetic construct
  source 1..41: mol_type = other DNA
ggaagatctagaggaaccacctaggacggtsascttggtcc

SEQ ID NO: 7 - DNA, 53 bp, synthetic construct
  source 1..53: mol_type = other DNA
cctttctatgcggcccagccggccgagctcgacatccrgdtgacccagtctcc

SEQ ID NO: 8 - DNA, 41 bp, synthetic construct
  source 1..41: mol_type = other DNA
ggaagatctagaggaaccacctttgatttccaccttggtcc

SEQ ID NO: 9 - DNA, 76 bp, synthetic construct
  source 1..76: mol_type = other DNA
ggtggttcctctagatcttcctcctctggtggcggtggctcgggcggtggtgggcaggtgcagctgcaggagtcsg

SEQ ID NO: 10 - DNA, 44 bp, synthetic construct
  source 1..44: mol_type = other DNA
cagtcattctcgacttactagttgaggagacrgtgaccagggtg

SEQ ID NO: 11 - DNA, 30 bp, synthetic construct
  source 1..30: mol_type = other DNA
cctttctatgcggcccagccggccgagctc

SEQ ID NO: 12 - DNA, 22 bp, synthetic construct
  source 1..22: mol_type = other DNA
cagtcattctcgacttactagt

SEQ ID NO: 13 - protein, 473 aa, synthetic construct
MGWSCIILFLVATATGVHSQVQLVQSGAEVKKPGASMKVSCKASGYTFAGYYIHWVRQAPGQGLEWMGSINPNSGKATYAQRFHGRVTLTREMSINTAYMELTRLTSDDTAIYYCARDATYSTSFSPRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 124 aa, synthetic construct
QVQLVQSGAEVKKPGASMKVSCKASGYTFAGYYIHWVRQAPGQGLEWMGSINPNSGKATYAQRFHGRVTLTREMSINTAYMELTRLTSDDTAIYYCARDATYSTSFSPRWFDPWGQGTLVTVSS

SEQ ID NO: 15 - protein, 8 aa, synthetic construct
GYTFAGYY

SEQ ID NO: 16 - protein, 8 aa, synthetic construct
INPNSGKA

SEQ ID NO: 17 - protein, 17 aa, synthetic construct
ARDATYSTSFSPRWFDP

SEQ ID NO: 18 - protein, 5 aa, synthetic construct
GYYIH

SEQ ID NO: 19 - protein, 17 aa, synthetic construct
SINPNSGKATYAQRFHG

SEQ ID NO: 20 - protein, 15 aa, synthetic construct
DATYSTSFSPRWFDP

SEQ ID NO: 21 - protein, 7 aa, synthetic construct
GYTFAGY

SEQ ID NO: 22 - protein, 6 aa, synthetic construct
NPNSGK

SEQ ID NO: 23 - protein, 6 aa, synthetic construct
AGYYIH

SEQ ID NO: 24 - protein, 13 aa, synthetic construct
WMGSINPNSGKAT

SEQ ID NO: 25 - protein, 16 aa, synthetic construct
ARDATYSTSFSPRWFD

SEQ ID NO: 26 - DNA, 1422 bp, synthetic construct
  source 1..1422: mol_type = other DNA
atgggatggtcatgtatcatcctttttctagtagcaactgcaaccggtgtacattcccaggtccagctggtacagtctggggctgaggtgaagaagcctggggcctcaatgaaggtctcctgcaaggcttctgggtacaccttcgccggctactatatacactgggtgcgacaggcccctggacaagggcttgagtggatgggatccatcaaccctaatagtggtaaagcaacctatgcacagaggtttcacggcagggtcaccttgaccagggagatgtccattaatacagcctacatggaactgaccaggctgacatctgacgacacggccatatactactgtgcgagagatgcgacatatagcaccagtttttcccctcgttggttcgacccctggggccaggggaccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 27 - protein, 233 aa, synthetic construct
MGWSCIILFLVATATGSWAEIVLTQSPGTLSLSPGERATLSCRASQSISSNSLAWYQQRPGQAPRLLIYGASSRATGIPDRFSGSGSGTDFTLTISRLEPEDFAVYYCQQYGSSPRTFGPGTKVDIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 108 aa, synthetic construct
EIVLTQSPGTLSLSPGERATLSCRASQSISSNSLAWYQQRPGQAPRLLIYGASSRATGIPDRFSGSGSGTDFTLTISRLEPEDFAVYYCQQYGSSPRTFGPGTKVDIK

SEQ ID NO: 29 - protein, 7 aa, synthetic construct
QSISSNS

SEQ ID NO: 30 - protein, 9 aa, synthetic construct
QQYGSSPRT

SEQ ID NO: 31 - protein, 12 aa, synthetic construct
RASQSISSNSLA

SEQ ID NO: 32 - protein, 7 aa, synthetic construct
GASSRAT

SEQ ID NO: 33 - protein, 8 aa, synthetic construct
SSNSLAWY

SEQ ID NO: 34 - protein, 10 aa, synthetic construct
LLIYGASSRA

SEQ ID NO: 35 - protein, 8 aa, synthetic construct
QQYGSSPR

SEQ ID NO: 36 - DNA, 702 bp, synthetic construct
  source 1..702: mol_type = other DNA
atgggatggtcatgtatcatcctttttctagtagcaactgcaaccggttcctgggccgaaattgtgttgacgcagtctccaggcaccctgtctttgtctccaggggaaagagccaccctctcctgcagggccagtcagagtattagcagcaactccttggcctggtaccagcagagacctggccaggctcccaggctcctcatctatggtgcatccagcagggccactggcatcccagacaggttcagtggcagtgggtccgggacagacttcactctcaccatcagcagactggagcctgaagattttgcagtgtattactgtcagcagtatggtagctcacctcgcactttcggccctgggaccaaagtggatatcaaaggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 37 - protein, 468 aa, synthetic construct
MGWSCIILFLVATATGVHSQVQLVQSGAEVKKPGASVKVSCRASGYTFTSHYIHWVRQAPGQGLEWMGITNPSTGSTNYAQQFQGRVTMTRDTSTSTVYMEVSSLRSEDTAIYYCARDVKRLGNLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 119 aa, synthetic construct
QVQLVQSGAEVKKPGASVKVSCRASGYTFTSHYIHWVRQAPGQGLEWMGITNPSTGSTNYAQQFQGRVTMTRDTSTSTVYMEVSSLRSEDTAIYYCARDVKRLGNLDYWGQGTLVTVSS

SEQ ID NO: 39 - protein, 8 aa, synthetic construct
GYTFTSHY

SEQ ID NO: 40 - protein, 8 aa, synthetic construct
TNPSTGST

SEQ ID NO: 41 - protein, 12 aa, synthetic construct
ARDVKRLGNLDY

SEQ ID NO: 42 - protein, 5 aa, synthetic construct
SHYIH

SEQ ID NO: 43 - protein, 17 aa, synthetic construct
ITNPSTGSTNYAQQFQG

SEQ ID NO: 44 - protein, 10 aa, synthetic construct
DVKRLGNLDY

SEQ ID NO: 45 - protein, 7 aa, synthetic construct
GYTFTSH

SEQ ID NO: 46 - protein, 6 aa, synthetic construct
NPSTGS

SEQ ID NO: 47 - protein, 6 aa, synthetic construct
TSHYIH

SEQ ID NO: 48 - protein, 13 aa, synthetic construct
WMGITNPSTGSTN

SEQ ID NO: 49 - protein, 11 aa, synthetic construct
ARDVKRLGNLD

SEQ ID NO: 50 - DNA, 1407 bp, synthetic construct
  source 1..1407: mol_type = other DNA
atgggatggtcatgtatcatcctttttctagtagcaactgcaaccggtgtacattcccaggtccagctggtgcagtctggggctgaggtgaagaagcctggggcctcagtgaaggtgtcctgcagggcatctggatacactttcaccagtcactatattcactgggtgcgacaggcccctggccaagggcttgagtggatgggaataactaatcctagtactggttccacaaactacgcacagcagttccagggcagagtcaccatgaccagggacacgtccacgagcacagtctacatggaggtgagcagcctgagatctgaggacacggcgatatattactgtgcgagagacgtgaagcgactggggaaccttgactactggggccagggaaccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 51 - protein, 235 aa, synthetic construct
MGWSCIILFLVATATGSWAQPVLTQPPSASGTPGQRVTISCSGSSSNIGRNYVYWYQQFPGTAPKLLIYRNNQRPSGVPDRFSASKSGTSASLAISGLRSEDEADYYCAAWDDRLSGAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - protein, 110 aa, synthetic construct
QPVLTQPPSASGTPGQRVTISCSGSSSNIGRNYVYWYQQFPGTAPKLLIYRNNQRPSGVPDRFSASKSGTSASLAISGLRSEDEADYYCAAWDDRLSGAVFGGGTQLTVL

SEQ ID NO: 53 - protein, 8 aa, synthetic construct
SSNIGRNY

SEQ ID NO: 54 - protein, 11 aa, synthetic construct
AAWDDRLSGAV

SEQ ID NO: 55 - protein, 13 aa, synthetic construct
SGSSSNIGRNYVY

SEQ ID NO: 56 - protein, 7 aa, synthetic construct
RNNQRPS

SEQ ID NO: 57 - protein, 9 aa, synthetic construct
IGRNYVYWY

SEQ ID NO: 58 - protein, 10 aa, synthetic construct
LLIYRNNQRP

SEQ ID NO: 59 - protein, 10 aa, synthetic construct
AAWDDRLSGA

SEQ ID NO: 60 - DNA, 708 bp, synthetic construct
  source 1..708: mol_type = other DNA
atgggatggtcatgtatcatcctttttctagtagcaactgcaaccggttcctgggcccagccagtgctgactcagccaccctcagcgtctgggacccccgggcagagggtcaccatttcttgttctggaagcagctccaacatcggaaggaattatgtatactggtatcagcagttcccaggaacggcccccaaactcctcatctataggaataatcagcggccctcaggggtccctgaccgattctctgcctccaagtctggtacctcagcctccctggccatcagtgggctccggtccgaggatgaggctgattattattgtgcagcatgggatgacagattgagtggcgctgtgttcggaggaggcacccagctgaccgtcctcggtcagcccaaggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 61 - protein, 4 aa, synthetic construct
GGGS